(19) United States
(12) Patent Application Publication
(10) Pub. No.: US 2026/0055431 A1
(43) Pub. Date: Feb. 26, 2026

(54) METHODS OF DELIVERING COMPONENTS TO MICROORGANOSPHERES

(71) Applicants: Shaun STEELE, Durham, NC (US); Marcin PADUCH, Durham, NC (US); Xilis, Inc., Durham, NC (US)

(72) Inventors: Shaun STEELE, Durham, NC (US); Marcin PADUCH, Durham, NC (US)

(21) Appl. No.: 19/104,350

(22) PCT Filed: Aug. 17, 2023
(86) PCT No.: PCT/US2023/072356

§ 371 Date: Feb. 17, 2025

Related U.S. Application Data
(60) Provisional application No. 63/371,693, filed on Aug. 17, 2022.

(51) Int. Cl. C12N 15/90; C12N 5/00; C12N 9/22; C12N 13/00; C12N 15/11; C12N 15/86; C12N 15/88

(52) U.S. Cl. CPC C12N 15/907; C12N 5/0062; C12N 9/226; C12N 13/00; C12N 15/11; C12N 15/86; C12N 15/88; C12N 2310/20; C12N 2510/00; C12N 2740/15043

(57) ABSTRACT

Systems and methods consistent with the present disclosure relate to MicroOrganoSpheres (MOSs). More particularly, methods relate to delivering components into a MOS. The methods also relate to delivering components into a MOS for screening drugs and biologics.

### CROSS REFERENCE TO RELATED APPLICATION

This application claims priority to U.S. Provisional Application No. 63/371,693 filed on Aug. 17, 2022. The content of the application is incorporated herein by reference in its entirety.

### FIELD OF THE DISCLOSURE

Systems and methods consistent with the present disclosure generally relate to MicroOrganoSpheres (MOSs). More particularly, in some embodiments, the methods relate to delivering components into a MOS. In particular, in some embodiments, methods relate to delivering gene editing components into a MOS to edit RNA or DNA comprised within a MOS. The methods also relate to delivering components into a MOS for screening drugs and biologics.

### Discussion of the Field

Model cell and tissue systems are useful for biological and medical research. The most common practice is to derive immortalized cell lines from tissue and culture them in two-dimensional (2D) conditions (e.g., in Petri dish or well plate). However, although immensely useful for basic research, 2D cell lines do not correlate well with individual patient response to therapy. In particular, three-dimensional cell culture models are proving particularly helpful in developmental biology, disease pathology, regenerative medicine, drug toxicity and efficacy testing, and personalized medicine. For example, spheroids and organoids are three-dimensional cell aggregates that have been studied. However, both traditionally formed organoids and spheroids have limitations that reduce their utility in certain applications.

Multicellular tumor spheroids were first described in the early 70s and obtained by culture of cancer cell lines under non-adherent conditions. Spheroids are typically formed from cancer cell lines as freely floating cell aggregates in ultra-low attachment plates. Spheroids have been shown to maintain more stem cell associated properties than 2D cell culture.

Organoids are in-vitro derived cell aggregates that include a population of stem cells that can differentiate into cells of major cell lineages. Organoids typically have a diameter of more than one mm, and are cultured through passages. It is typically slower to grow and expand organoid culture than 2D cell culture. To generate organoids from clinical samples, requires a sufficient number of viable cells (e.g., hundreds to thousands) to start with, so it is often challenging to derive organoids from low volume samples, such as from a biopsy, and even if successful—it would take considerable time to expand the culture for applications such as drug testing. In addition, there is a large amount of variability in organoid size, shape and cell number. Organoids may require complex cocktails of growth factors and culture conditions in order to grow and express desired cell types.

Neither tumor spheroids nor organoids are optimal for rapid and reliable screening, particularly for personalized medicine, such as performing ex-vivo testing of drug response. For example, the practice of oncology continually faces an immense challenge of matching the right therapeutic regimen with the right patient, in addition to balancing relative benefit with risk to achieve the most favorable outcome. Patient-Derived Models of Cancer (PDMC) may include the use of organoids (including patient-derived organoids) to facilitate the identification and development of more individualized therapeutic targets. However, although retrospective studies have shown that organoids derived from resected or biopsied patient tumors correlate with patient response to therapy, there are major limitations in using organoids to guide therapy. As mentioned above, it takes months to derive and expand organoids, and particularly patient-derived organoids, from tumor samples for drug sensitivity tests, which decreases the clinical applicability, as patients cannot wait that long to receive treatment. In addition, the number of organoids needed to perform a drug screen with more than dozens of compounds currently cannot be obtained in a clinically feasible timeframe from a core biopsy specimen, which is often the only available form of tissue from patients with metastatic or inoperable cancer. The significant failure rate for deriving organoids from biopsies also prevents its use as a reliable diagnostic assay. Further, there may be a high degree of variability in the size (and potentially the response) of organoids, particularly with longer culture times, and therefore many passages.

Due to their better correlations with patient outcomes, PDMCs are also being exploited to replace 2D cell lines as a high-throughput screen platform for drug discovery, such as RNAi, CRISPR, and pharmacological small molecule screens. However, compared to cell lines, these PDMC models (including spheroids and organoids) are typically much slower to expand and manipulate, making it challenging and costly for high-throughput applications. The longer time required to expand these models to amplify the cell numbers also tends to allow the fastest growing clone in plastics to dominate and outcompete other clones, hence making the model more homogeneous and losing the original tissue compositions and clonal diversity. Furthermore, their relatively larger and heterogeneous sizes and limited diffusibility make them challenging for many automated fluorescence and imaging-based readout assays.

Methods, compositions and apparatuses for generating MOSs that are patient derived tissue models (e.g., tumor models and/or non-tumor tissue models) from resection or biopsies have previously been described. In particular, the methods and apparatuses previously described enable generation of a large number of patient-derived tissue models having predictable and clinically relevant properties from a single biopsy, such as an 18-gauge core biopsy, which can be completed within, e.g., 7-10 days after obtaining a biopsy. This permits robust and reliable testing and minimizes delays in guiding patient-specific therapies. Furthermore, such MOSs expand quickly in a highly parallel manner, generating units with smaller and more uniform sizes, allowing better control over cell number per unit, and better diffusibility (e.g., via increase surface to volume ratio), for high-throughput screening applications. Such MOSs are useful models for testing drugs and biopharmaceuticals to give a more accurate indication of individual patient responses to such therapies

What is further needed are methods for delivering components into a MOS. In particular, methods for delivering components useful in further assays and drug screening methods. In particular, methods for high throughput screening are needed.

### SUMMARY

Provided herein are methods of delivering one or more components into a MOS. In particular, in some embodiments, the methods relate to delivering gene editing components into a MOS. The methods also relate to editing DNA or RNA comprised within a MOS. Further methods include methods of drug screening in a MOS, and drug screening in a MOS comprising edited DNA or RNA, in particular high throughput methods. Also provided herein are MOSs obtained by the methods, provided herein, of delivering components into a MOS.

As illustrated in FIG. 43A, previous methods are based on introducing components, e.g. gene editing components, into individual dissociated cells, followed by generation of a 3D tissue model. FIG. 43B illustrates the methods provided herein in which 3D tissue models are generated as a MOS and components are introduced into the MOS.

Also provided herein, are methods of high throughput screening as illustrated in FIG. 44. MOSs are loaded into a high throughput format, for example a multiwell plate, and different components can be added to each well.

Furthermore, the methods provided herein overcome the challenges with delivery of components to exiting 3D tissue models such as patient derived organoids (PDOs), where it is difficult to introduce components such that they sufficiently penetrate into the tissue model.

Tissues and organs are multicellular structures that self organize in three dimensions (3D). Cells within a tissue interact with neighboring cells and with extracellular matrix (ECM) through biochemical and mechanical cues that maintain specificity and homeostasis of biological tissues. While traditional 2D cultures on rigid surfaces fail to reproduce in vivo cell behavior, 3D matrices are becoming increasingly popular supports for cell cultures because they allow mimicking the complex environment that supports cell physiological functions to better predict in vivo responses and thus to limit the need for animal models.

RNA interference (RNAI) and plasmid transfection have been widely used as powerful tools to alter the expression of specific genes and to observe resulting phenotypic changes. While nucleic acid transfection is highly effective in the majority of mammalian cells cultured under standard 2D conditions, additional obstacles are encountered for transfection of solid tissues or 3D models. Indeed, one limitation is that organoids are embedded in ECM, which constitutes a barrier for efficient transfection. Moreover, organoids grow into dense and compact structures that impede diffusion, penetration, and cellular accumulation of genetic material, which makes transfection via traditional techniques difficult. In addition, cells that are located at the center of 3D structures are often difficult to transfect, and so direct transfection of already formed organoids is challenging. This is particularly challenging when introducing CRISPR/Cas components which are larger and have a complex 3D structure that must be maintained.

The present invention provides methods for the delivery of components into MOS, including gene editing components such as CRISPR/Cas. The methods allow for gene editing at efficiencies of about 80%, or greater than 80%, or greater than 90% while maintaining cell viability and allowing the development of a 3D microenvironment and/or development into organoids and/or tissue models.

### MOS Formation and Applications

Described herein are MicroOrganoSpheres (MOSs), apparatuses and methods of making MOSs, and apparatuses and methods of using MOSs. Also described herein are methods and systems for screening a patient using these MOSs, including personalized therapy methods.

In general, described herein are methods and apparatuses that form and grow MOSs containing cells originating from a patient, for example, extracted from a small patient biopsy, (e.g., for quick diagnostics to guide therapy), from resected patient tissue, including resected primary tumor or part of a dysfunctional organ (e.g., for high-throughput screening), and/or from already established PDMCs, including patient-derived xenografts (PDX) and organoids (e.g., to generate MOSs for high-throughput screening).

These MOSs may be formed from primary cells that are normal (e.g., normal organ tissue) or from tumor tissue. For example, these methods and apparatuses may form MOSs from cancerous tumor biopsy tissue, enabling tailored treatments that can selected using the particular tumor tissue examined. Surprisingly, these methods and apparatuses permit the formation of hundreds, thousands or even tens of thousands (e.g., 500, 750, 1000, 2000, 5000, 10,000 or more) of MOSs from a single tissue biopsy, within a few hours of the biopsy being removed from the patient. Dissociated primary cells from the patient biopsy may be combined with a fluid matrix material, such as a substrate basement membrane matrix (e.g., MATRIGEL), to form the MOS. The resulting plurality of MOSs may have a predefined range of sizes (such as diameters, e.g., from 10 μm to 700 μm and any sub-range therewithin), and initial number of primary cells (e.g., between 1 and 1000, and in particular lower numbers of cells, such as between 1-200). The number of cells and/or the diameter may be controlled within, e.g., +/−5%, 10%, 15%, 20%, 25%, 30%, etc. These MOSs, when formed as described herein, have an exceptionally high survival rate (>75%, >80%, >85%, >90%, >95%) and are stable for use and testing within a very short period of time, including within the first 1-10 days after being formed (e.g., within 1 day, within 2 days, within 3 days, within 4 days, within 5 days, within 6 days, within 7 days, within 8 days, within 9 days, within 10 days, etc.). This allows for rapid tests on a potentially huge number of patient-specific and biologically relevant MOSs which may save critical time in developing and deploying a patient therapy, such as a cancer treatment plan. The MOSs described herein rapidly form a 3D microenvironment, which are 3D cellular structures that replicate and correspond to the tissue environment from which they were biopsied, such as a three-dimensional (3D) tumor microenvironment (tumorspheres, contained within the MOS which have organoid properties). The MOSs described herein may also be referred to as “droplets”. Each MOSs may include, e.g., as part of the fluid matrix material, growth factors and structural proteins (e.g., collagen, laminin, nidogen, etc.) that may mimic the original tissue (e.g., tumor) environment. Each MOS may also include immune cells of the original tissue. Virtually any primary cell tissue may be used, including virtually any tumor tissue.

For example, to date, all tumor types and sites tested have successfully produced MOSs (e.g., current success rate of 100%, n=32, including cancer of the colon, esophagus, skin (melanoma), uterus, bone (sarcoma), kidney, ovary, lung, and breast from the primary site or metastatic sites including liver, (omentum, and diaphragm). The tissue types used to successfully generate MOSs may be metastasized from other locations. The MOSs described herein can be grown from fine needle aspirate (FNA) or from circulating tumor cells (CTCs), e.g., from a liquid biopsy. Proliferation and growth are typically seen in as few as 3-4 days, and the MOSs can be maintained and passaged for months, or they may be cryopreserved and/or used for assays immediately (e.g., within the first 7-10 days).

In particular, described herein are methods of forming Patient-Derived MOSs. These methods may include combining dissociated primary tissue cells (including, but not limited to cancer/abnormal tissue, normal tissue, etc.) with a liquid matrix material to form an unpolymerized material, and then polymerizing the unpolymerized material to form MOSs that are typically less than about 1000 μm (e.g., less than about 900 μm, less than about 800 μm, less than about 700 μm, less than about 600 μm, and in particular, less than about 500 μm) in diameter in which the dissociated primary tissue cells are distributed. The number of dissociated cells may be within a predetermined range, as mentioned above (e.g., between about 1 and about 500 cells, between about 1-200 cells, between about 1-150 cells, between about 100 cells, between about 1-75 cells, between about 1-50 cells, between 35 about 1-30 cells, between about 1-20 cells, between about 1-10 cells, between about 5-15 cells, between about 20-30 cells, between about 30-50 cells, between about 40-60 cells, between about 50-70 cell, between about 60-80 cells, between about 70-90 cells, between about 80-100 cells, between about 90-110 cells, etc., including about 1 cell, about 10 cells, about 20 cells, about 30 cells, about 40 cells, about 50 cells, about 60 cells, about 70 cells, etc.). Any of these methods may be configured as described herein to produce MOSs of repeatable size (e.g., having a narrow distribution of sizes), as well as MOS that include immune cells.

The dissociated cells may be freshly biopsied and may be dissociated in any appropriate manner, including mechanical and/or chemical dissociation (e.g., enzymatic disaggregation by using one or more enzymes, such as collagenase, trypsin, etc.). The dissociated cells may optionally be treated, selected and/or modified. For example, the cells may be sorted or selected to identify and/or isolate cells having one or more characteristics (e.g., size, morphology, etc.). The cells may be marked (e.g., with one or more markers) that may be used to aid in selection. The cells may be sorted by a known cell-sorting technology, including but not limited to microfluidic cell sorting, fluorescent activated cell sorting, magnetic activated cell sorting, etc. Alternatively, the cells may be used without sorting.

Before MOS formation, the dissociated cells may be modified by treatment with one or more agents. For example, the cells may be genetically modified. The cells may be modified using CRISPR/Cas9 or other genetic editing techniques. The cells may be transfected by any appropriate method (e.g., electroporation, cell squeezing, nanoparticle injection, magnetofection, chemical transfection, viral transfection, etc.), including transfection with of plasmids, RNA, siRNA, etc. Alternatively, the cells may be used without modification.

One or more additional materials may be combined with the dissociated cells and fluid (e.g., liquid) matrix material to form the unpolymerized mixture. For example, the unpolymerized mixture may include additional cell or tissue types, including support cells. The additional cells or tissue may originate from different biopsy (e.g., primary cells from a different dissociated tissue) and/or cultured cells. The additional cells may be, for example immune cells, stromal cells, endothelial cells, etc. The additional materials may include medium (e.g., growth medium, freezing medium, etc.), growth factors, support network molecules (e.g., collagen, glycoproteins, extracellular matrix, etc.), or the like. The additional materials may include a drug composition. The unpolymerized mixture includes only the dissociated tissue sample (e.g., primary cells) and the fluid matrix material.

The methods may rapidly form a plurality of MOSs from a single tissue biopsy, so that greater than about 500 Patient-Derived MOSs are formed from per biopsy (e.g., greater than about 600, greater than about 700, greater than about 800, greater than about 900, greater than about 1000, greater than about 2000, greater than about 2500, greater than about 3000, greater than about 4000, greater than about 5000, greater than about 6000, greater than about 7000, greater than about 8000, greater than about 9000, greater than about 10,000, greater than about 11,000, greater than about 12,000, etc.). The biopsy may be a standard size biopsy, such as an 18G (e.g., 14G, 16G, 18G, etc.) core biopsy. For example, the volume of tissue removed by biopsy and used to form the plurality of MOSs may be a small cylinder (taken with a biopsy needle) of between about 1/32 and ⅛ of an inch diameter and about ¾ inch to ¼ inch long, such as a cylinder of about 1/16 inch diameter by ½ inch long. The biopsy may be taken by needle biopsy, e.g., by core needle biopsy. The biopsy may be taken by fine needle aspiration. Other biopsy types that may be used include shave biopsy, punch biopsy, incisional biopsy, excisional biopsy, and the like. Typically the material from a single patient biopsy may be used to generate the plurality (e.g., greater than about 2000, greater than about 5000, greater than about 7500, greater than about 10,000, etc.) of MOSs, as described above. The plurality of Patient-MOSs may be formed using an apparatus (as described herein) that may be configured to generate this large number of highly regular (size, cell number, etc.) MOSs as described herein. The methods and apparatuses may generate the plurality or MOSs at a rapid rate (e.g., greater than about 1 MOS per minute, greater than about 1 MOS per 10 seconds, greater than about 1 MOS per 5 seconds, greater than about 1 MOS per 2 seconds, greater than about 1 MOS per second, greater than about 2 MOSs per second, greater than about 3 MOSs per second, greater than about 4 MOSs per second, greater than about 5 MOSs per second, greater than about 10 MOSs per second, greater than 50 MOSs per second, greater than 100 MOSs per second, greater than 125 MOSs per second, etc.).

For example, these methods may be performed by combing the unpolymerized mixture with a material (e.g., liquid material) that is immiscible with the unpolymerized material. The method and apparatus may control the size and/or cell density of the MOSs by, at least in part, controlling the flow of one or more of the unpolymerized mixture (and/or the dissociated tissue and fluid matrix) and the material that is immiscible with the unpolymerized mixture (e.g., a hydrophobic material, oil, etc.). For example, these methods may be performed using a microfluidics apparatus. Multiple MOSs may be formed in parallel (e.g., 2 in parallel, 3 in parallel, 4 in parallel, etc.). The same apparatus may therefore include multiple parallel channels, which may be coupled to the same source of unpolymerized material, or the same source of dissociated primary tissue and/or a source of fluid matrix.

The unpolymerized material may be polymerized in order to form the MOSs in a variety of different ways. The methods may include polymerizing the MOSs by changing the temperature (e.g., raising the temperature above a threshold value, such as, for example greater than about 20 degrees C., greater than about 25 degrees C., greater than about 30 degrees C., greater than about 35 degrees C., etc.).

Once polymerized, the MOSs may be allowed to grow, e.g., by culturing and/or may be assayed either before or after culturing and/or may be cryopreserved either before or after culturing. The MOSs may be cultured for any appropriate length of time, but in particular, may be cultured for between 1 day and 10 days (e.g., between 1 day and 9 days, between 1 day and 8 days, between 1 day and 7 days, between 1 day and 6 days, between 3 days and 9 days, between 3 days and 8 days, between 3 days and 7 days, etc.). The MOSs may be cryopreserved or assayed before six passages, which may preserve the heterogeneity of the cells within the MOSs; limiting the number of passages may prevent the faster-dividing cells from outpacing more slowly dividing cells.

In general, since the same patient biopsy may provide a high number (e.g., greater than 2,000, greater than 3,000, greater than 4,000, greater than 5,000, greater than 6,000, greater than 7,000, greater than 8,000, greater than 9,000, greater than 10,000, etc.) cells, some of the MOSs may be cryopreserved (e.g., over half) while some are cultured and/or assayed. As will be described in greater detail herein, cryopreserved MOSs may be banked and used (e.g., assayed, passaged, etc.) later.

Thus, described herein are methods, including methods of forming a plurality of MOSs. For example, a method of forming a plurality of MOSs may include: combining a dissociated tissue sample and a fluid matrix material to form an unpolymerized mixture; forming a plurality of droplets of the unpolymerized mixture; and polymerizing the droplets to form a plurality of MOSs each having a diameter of between 50 and 500 μm with between 1 and 200 dissociated cells distributed therein.

A method, e.g., of forming a plurality of MOSs, may include combining a dissociated tissue sample and a fluid matrix material to form an unpolymerized mixture; forming a plurality of droplets from a continuous stream of the unpolymenzed mixture wherein the droplets have less than a 25% embodiment in size; and polymerizing the droplets by warming to form a plurality of MOSs each having between 1 and 200 dissociated cells distributed within each MOS.

A method as described herein for forming a plurality of MOSs may include: combining a dissociated tissue sample and a fluid matrix material to form an unpolymerized mixture; forming a plurality of droplets having less than a 25% embodiment in size of the droplets by converging a stream of the unpolymerized mixture with one or more streams of a fluid that is immiscible with the unpolymerized mixture; polymerizing the droplets to form a plurality of MOSs having a diameter of between 50 and 500 μm with between 1 and 200 dissociated cells distributed therein; and separating the plurality of MOSs from the fluid that is immiscible.

Any of these methods may include modifying the cells within the dissociated tissue sample prior to forming the droplets.

Forming the plurality of droplets may comprise forming a plurality of droplets of the unpolymerized mixture of uniform size with less than about 25% embodiment in size (e.g., less than about 20% embodiment in size, less than about 15% embodiment in size, less than about 10% embodiment in size, less than about 8% embodiment in size, less than about 5% embodiment in size, etc.). The embodiments in size may also be described as a narrow distribution of size embodiment. For example, the distribution of sizes may include a MOS size distribution (e.g., MOS diameter vs. the number of formed MOSs) having a low standard deviation (e.g., a standard deviation of 15% or less, a standard deviation of 12% or less, a standard deviation of 10% or less, a standard deviation of 8% or less, a standard deviation of 6% or less, a standard deviation of 5% or less, etc.).

Any of these methods may also include plating or distributing the MOSs. For example, the method may include combining MOSs from various sources into a receptacle prior to assaying. For example, the MOSs may be placed into a multi-well plate. Thus, any of these methods may include dispensing the MOSs into a multi-well plate prior to assaying the MOSs. One or more (or equal amounts of) MOSs may be included per well.

Applying the MOSs into a receptacle may include placing the MOSs into a plurality of chambers that are separated by an at least partially permeable membrane to permit circulation of supernatant material between the chambers. This may allow the MOSs to share the same supernatant.

In any of these methods the MOSs may be assayed. An assay may generally include exposing or treating individual MOSs to a condition (e.g., drug compositions or combinations of drug compositions, including but not limited to any of the drug compositions described herein) to determine if the condition has an effect on the cells of the MOSs (and in some cases, what effect it has). Assays may include exposing a subset of the MOSs (individually or in groups) to one or more concentrations of a drug composition, and allowing the MOSs to remain exposed for a predetermined time period (minutes, hours, days, etc.) and/or exposing and removing the drug composition, then culturing the MOSs for a predetermined time period. Thereafter the MOSs may be examined to identify any effects, including in particular toxicity on the cells in the MOSs, or a change in morphology and/or growth of the cells in the MOSs. Assaying may include marking (e.g., by immunohistochemistry) live or fixed cells within the MOSs. Cells may be assayed (e.g., examined) manually or automatically. For example, cells may be examined to determine any toxicity (cell death) using an automated reader apparatus. Assaying the plurality of MOSs may include sampling one or more of a supernatant, an environment, and a microenvironment of the MOSs for secreted factors and other effects. The MOSs may be recovered following the assay for further assaying, expansion or preservation (e.g., cryopreserving, fixation, etc.) for subsequent examination.

As mentioned, virtually any assay may be used. For example, genomic, transcriptomic, proteomics, or meta-genomic markers (such as methylation) may be assayed using the MOSs described herein. Thus, any of these compositions and methods described herein may be used to identify or examine one or more markers and biological/physiological pathways, including, for example, exosomes, which may assist in identifying drugs and/or therapies for patient treatment.

Any appropriate tissue sample may be used. The tissue sample may include a biopsy sample from a metastatic tumor. For example, a tissue sample may comprise a clinical tumor sample; the clinical tumor sample may comprise both cancer cells and stroma cells. The tissue sample may comprise tumor cells and one or more of: mesenchymal cells, endothelial cells, and immune cells.

Any of the methods described herein may include initially distributing the dissociated cells from the tissue biopsy uniformly, or non-uniformly, throughout the fluid matrix material, in any appropriate concentration. For example, the methods described herein may include combining the dissociated tissue sample and the fluid matrix material so that the dissociated tissue cells are distributed within the fluid matrix material to a density of less than 1×107 cells/mi (e.g., less than 9×106 cells/ml, 7×106 cells/ml, 5×106 cells/ml, 3×106 cells/ml, 1×106 cells/ml, 9×105 cells/ml, 7×105 cells/ml, 5×105 cells/ml, etc.).

In general, forming the droplet may comprise forming the droplet from a continuous stream of the unpolymerized mixture. For example, forming the droplet may comprise applying one or more convergent streams of a fluid that is immiscible with the unpolymerized mixture to the stream of unpolymerized mixture. The streams may be combined in a microfluidic device, e.g., a device having a plurality of converging channels into which the unpolymerized mixture and the immiscible fluid interact to form droplets having a precisely controlled volume. The droplets may be formed (e.g., pinched off) in an excess of the immiscible material, and the droplets may be concurrently and/or subsequently polymerized to form the MOSs. For example, the region in which the streams converge may be configured to polymerize the unpolymerized mixture after the droplet has been formed, e.g., by heating, and/or the regions downstream may be configured to polymerize the unpolymerized mixture after the droplets have been formed and are surrounded by the immiscible material. The immiscible material may be heated (or alternatively cooled) to a temperature that promotes polymerization of the unpolymerized material, forming the MOSs. For example, polymerizing may comprise heating the droplet to greater than 35 degrees C.

Thus, in any of these methods, forming the droplet may include forming the droplet in a fluid that is immiscible with the unpolymerized mixture. Further, any of these methods may include separating the immiscible fluid from the MOSs. Further, any of these methods may include removing the immiscible fluid from the MOSs. In general, an immiscible fluid may include a liquid (e.g., oil, polymer, etc.), including in particular a hydrophobic material or other material that is immiscible with the unpolymerized (e.g., aqueous) material.

The fluid matrix material may be a synthetic or non-synthetic unpolymerized basement membrane material. The unpolymerized basement material may comprise a polymeric hydrogel. The fluid matrix material may comprise a MATRIGEL. Thus, combining the dissociated tissue sample and the fluid matrix material may comprise combining the dissociated tissue sample with a basement membrane matrix.

The tissue sample may be combined with the fluid matrix material within six hours of removing the tissue sample from the patient or sooner (e.g., within about 5 hours, within about 4 hours, within about 3 hours, within about 2 hours, within about 1 hour, etc.).

Also described herein are methods of assaying or preserving MOSs. For example, a method may include: combining a dissociated tissue sample and a fluid matrix material to form an unpolymerized mixture; forming a plurality of droplets of the unpolymerized mixture having less than a 25% embodiment in a size of the droplets; polymerizing the droplets to form a plurality of MOSs having a diameter of between 50 and 700 μm with between 1 and 1000 dissociated cells distributed therein; and assaying or cryopreserving the plurality of MOSs.

The methods may include: combining a dissociated tissue sample and a fluid matrix material to form an unpolymerized mixture; forming a plurality of droplets of the unpolymerized mixture; polymerizing the droplets to form a plurality of MOSs each having a diameter of between 50 and 500 μm with between 1 and 200 dissociated cells distributed therein; and cryopreserving or assaying the plurality of MOSs within 15 days, wherein the MOSs are assayed to determine the effect of one or more agents on the cells within the MOSs.

For example, a method may include: combining a dissociated tissue sample and a fluid matrix material to form an unpolymerized mixture; forming a plurality of droplets having less than a 25% embodiment in a size of the droplets by converging a stream of the unpolymerized mixture with one or more streams of a fluid that is immiscible with the unpolymerized mixture; polymerizing the droplets by warming to form MOSs each having a diameter of between 50 and 500 μm with between 1 and 200 dissociated cell distributed therein; and assaying or cryopreserving the MOSs before six passages, whereby heterogeneity of the cells within the MOSs is maintained, further wherein assaying comprises assaying in order to determine the effect of one or more agents on the cells within the MOSs.

In any of these methods, the plurality of MOSs may be cryopreserved or assayed before six passages, whereby heterogeneity of the cells within the MOSs is maintained. Any of these methods may further include modifying the cells within the dissociated tissue sample prior to forming the droplets.

Forming the droplet may include forming a plurality of droplets of the unpolymerized mixture of uniform size with less than about 25% embodiment in size (e.g., less than about 20%, less 35 than about 15%, less than about 10%, less than about 7%, less than about 5%, etc.).

Any of these methods may include culturing the MOSs for an appropriate length of time, as mentioned above (e.g., culturing the MOSs for between 2-14 days before assaying). For example, these methods may include removing the immiscible fluid from the MOSs before culturing. Culturing the MOSs may comprise culturing the MOSs in suspension.

In general, assaying the MOSs may comprise genomically, transcriptomically, epigenomically and/or metabolically analyzing the cells in the MOSs before and/or after assaying or cryopreserving the MOSs. Any of these methods may include assaying the MOSs by exposing the MOSs to a drug (e.g., drug composition).

In any of these methods, assaying may comprise visually assaying the effect of the one or more agents on the cells in the MOSs either manually and/or automatically. Any of these methods may include marking or labeling cells in the MOSs for visualization. For example, assaying may include fluorescently assaying the effect of the one or more agents on the cells.

The MOSs described herein are themselves novel and may be characterized as a composition of matter. For example, a composition of matter may comprise a plurality of cryopreserved MOSs, wherein each MOS has a spherical shape having a diameter of between 50 μm and 500 μm and comprises a polymerized base material, and between about 1 and 1000 dissociated primary cells distributed within the base material that have been passaged less than six times, whereby heterogeneity of the cells within the MOSs is maintained. The MOSs may include cells of the immune system (“immune cells”).

Also described herein are compositions of matter comprising a plurality of cryopreserved MOSs, wherein each MOS has a spherical shape having a diameter of between 50 μm and 500 μm, wherein the MOSs have less than a 25% embodiment in size, and wherein each MOS comprises a polymerized base material, and between about 1 and 500 dissociated primary cells distributed within the base material that have been passaged less than six times, whereby heterogeneity of the cells within the MOSs is maintained. The MOSs may include immune cells from the tissue of origin.

The primary cells may be primary tumor cells. For example, the dissociated primary cells may have been genetically or biochemically modified. The plurality of cryopreserved MOSs may have a uniform size with less than 25% embodiment in size. The plurality of cryopreserved MOSs may comprise MOSs from various sources. In any of these MOSs, the majority of cells in each MOS may comprise cells that are not stem cells. The primary cells may comprise metastatic tumor cells. The primary cells may comprise both cancer cells and stroma cells. The primary cells may comprise tumor cells and one or more of: mesenchymal cells, endothelial cells, and immune cells.

The primary cells may be distributed within the polymerized base material at a density of less than, e.g., 5×107 cells/ml, 1×107 cells/ml, 9×106 cells/ml, 7×106 10 cells/ml, 5×106 cells/ml. 1×106 cells/ml, 9×105 cells/ml, 7×105 cells/ml, 5×105 cells/ml, 1×105 cells/ml, etc.

In general, the polymerized base material may comprise a basement membrane matrix (e.g., MATRIGEL). The polymerized base material may comprise a synthetic material.

The microoganoids may have a diameter of between 50 μm and 1000 μm, or more preferably between 60 μm and 700 μm, or more preferably between 50 μm and 500 μm, or between 50 μm and 400 μm, or between 50 μm and 300 μm, or between 50 μm and 250 μm, etc. (e.g., less than about 500 μm, less than about 400 μm, less than about 300 μm, less than about 250 μm, less than about 200 μm, etc.).

As mentioned, the MOSs described herein may include any appropriate number of primary tissue cells initially in each MOS, for example less than about 200 primary cells, or more preferably less than about 150 primary cells, or more preferably less than about 100 primary cells, or more preferably less than about 75 primary cells, or less than about 50 cells, or less than about 30 cells, or less than about 25 cells, or less than about 20 cells or less than about 10 cell, or less than about 5 cells, etc.). Each MOS may include between about 1 and 500 cells, between about 1-400 cells, between bout 1-300 cells, between about 1-200 cells, between about 1-150 cells, between about 1-100 cells between about 1-75 cells, between about 30 1-50 cells, between about 1-30 cells, between about 1-25 cells, between about 1-20 cells, etc.

Also described herein are apparatuses for forming MOSs, and methods of operating these apparatuses to form the MOSs. For example, described herein are methods of operating a MOS forming apparatus comprising: receiving an unpolymerized mixture comprising a chilled mixture of a dissociated tissue sample and a first fluid matrix material in a first port; receiving a second fluid that is immiscible with the unpolymerized mixture in a second port; combining a stream of the unpolymerized mixture with one or more streams of the second fluid to form droplets of the unpolymerized mixture having a uniform size that varies by less than 25%; and polymerizing the droplets of the unpolymerized mixture to form a plurality of MOSs.

A method of operating a MOS forming apparatus may include: receiving an unpolymerized mixture comprising a chilled mixture of a dissociated tissue sample and a first fluid matrix material in a first port; receiving a second fluid that is immiscible with the unpolymerized mixture in a second port; combining a stream of the unpolymerized mixture at a first rate with one or more streams of the second fluid at a second rate to form droplets of the unpolymerized mixture having a uniform size that varies by less than 25%, wherein the droplets are between 50 μm and 500 μm diameter; and polymerizing the droplets of the unpolymerized mixture to form a plurality of MOSs.

Any of these methods may include coupling a first reservoir containing the unpolymerized mixture in fluid communication with the first port. For example, the method may include combining the dissociated tissue sample and the first fluid matrix material to form the unpolymerized mixture. The method may include adding the unpolymerized mixture to a first reservoir in fluid communication with the first port. These methods may include coupling a second reservoir containing the second fluid in fluid communication with the second port. Any of these methods may include adding the second fluid to a second reservoir in fluid communication with the second port. Receiving the second fluid may comprise receiving an oil.

In general, these methods may include separating the second fluid (e.g. the immiscible fluid) from the plurality of MOSs. This fluid may be manually or automatically separated. For example, the second (immiscible) fluid may be removed by washing, filtering, or any other appropriate method.

Combining the streams may comprise driving the stream of the unpolymerized mixture at a first flow rate across one or more streams of the second fluid which is traveling at a second flow rate. The first flow rate may be greater than the second flow rate. Either or both the flow rate and/or the amount of material (e.g., the unpolymerized mixture) may be present in smaller amount than the second fluid, so that the unpolymerized mixture is encapsulated in a precisely-controlled droplet, as described herein, that may then be polymerized, e.g., within the second fluid.

Combining the streams may comprise driving the stream of the unpolymerized mixture across a junction into which the one or more streams of the second fluid also converge. Polymerizing the droplets may comprise heating the droplets to greater than a temperature at which the unpolymerized material polymerizes (e.g., greater than about 25 degrees C., greater than about 30 degrees C., greater than about 35 degrees C., etc.).

Any of these methods may include aliquoting the plurality of MOSs. For example, aliquoting into a multi-well dish.

Also described herein are methods of treating a patient using these MOSs, and methods of assaying them. For example a method may include: receiving a patient biopsy from a tumor; and determining, within 2 weeks of taking of the biopsy, that the tumor will respond to a drug formulation by: forming, from the patient biopsy, a plurality of MOSs having a diameter of between 50 and 500 μm with between 1 and 200 dissociated tumor cells distributed through a polymerized base material, and exposing at least some of the MOSs to the drug formulation before the dissociated tumor cells have undergone more than five passages; and measuring an effect of the drug formulation on the cells within the at least some of the MOSs to determine if the drug will treat the tumor based on the determined effect.

These methods may include determining that the tumor is still responding to the drug formulation after one or more administrations of the drug to the patient by receiving a second patient biopsy after the patient has been treated with the drug formulation and forming a second plurality of MOSs from the second patient biopsy, exposing at least some of the second plurality of MOSs to the drug formulation, and measuring the effect of the drug formulation on cells within the at least some of the second plurality of MOSs.

Determining that the tumor will respond to a drug formulation may include exposing at least some of the MOSs to a plurality of drug formulations, and reporting the measured effects for each of the drug formulations. Determining may further comprise dispensing the MOSs into a multi-well plate prior to assaying the MOSs.

Any of these methods may include biopsying the patient to collect the patient biopsy (or otherwise taking a tissue sample from a patient or a sample of patient-derived tissues or cells) and/or treating the patient with the drug formulation, or assisting a physician in treating the patient (e.g., advising the physician as to which drug formulations would be effective). In general, the time between receiving the biopsy and reporting may be less than about 21 days (e.g., less than about 15 days, less than about 14 days, less than about 13 days, less than about 12 days, less than about 11 days, less than about 10 days, less than about 9 days, less than about 8 days, less than about 7 days, etc.).

MOSs can be used for testing certain therapies that were previously difficult to test. Unlike in the formation of traditional bulk organoids, cells of the immune system (“immune cells”) present in the biopsied patient derived tissues (e.g., from a tumor) may also be present and persist in MOSs upon their formation, even after the extensive processing for MOS formation described herein. Immune cells in MOS prepared as described herein can persist for 7 days or more, and in some cases 14 days or more. Immune cells may persist for 21 days or longer. Additionally, when using traditional bulk organoids, it can be difficult for some therapies, such as certain immune-oncology therapies and T cell biopharmaceuticals, to penetrate, reach and interact with patient derived tissues (e.g., from a tumor) within. In contrast, MOSs allow for penetration of those drugs much more readily.

Because MOS formation as described herein allow for immune cells from the patient derived tissues to be incorporated, the accuracy of testing the aforementioned drug formulations in MOSs is superior to testing in traditional bulk organoids. In addition, immune cells derived from a patient may be separately introduced into MOS that have already formed, because of ease of penetration. Patient derived tissues (e.g., from a tumor) will include a variety of immune cells that are natively produced by the patient's body. A patient's response to particular drug formulations (e.g., immune-oncology drugs and biologics) can be directly impacted by the immune cells present at the target site. MOSs produced as described herein can be advantageous for such drug testing, as the drugs described herein can be tested in MOSs that include immune cells from the tissue of origin upon formation, or MOSs that include immune cells that are introduced after MOSs are formed.

It may be desirable to utilize therapies derived from a patient's own immune cells. For example, autologous immune enhancement therapy allows immune cells (i) to be taken out from a patient's body, (ii) to be cultured and processed to activate them until their resistance to, for example, cancer is strengthened, and (iii) to be put back in the patient's body. Because such enhanced immune cells do not readily penetrate traditional bulk organoids, it can be difficult to test the efficacy of enhanced immune cells in vitro. However, the size and composition of MOSs are able to uptake such enhance immune cells. Accordingly, MOSs can be used to test enhanced immune cells for efficacy in a patient and reduce the risk of subjecting patients to ineffective immune cell infusions and supplemental immune cell harvesting for additional infusions.

### BRIEF DESCRIPTION OF THE DRAWINGS

The accompanying drawings, which are incorporated in and constitute a part of this disclosure, illustrate various, methods, embodiments and aspects of the present invention and background to the invention. In the drawings:

FIGS. 1A to 1C illustrate Patient-Derived MOSs formed as described herein to include a single dissociated primary tissue cell per MOS, cultured for one day after forming (FIG. 1A), cultured for three days after forming (FIG. 1B), and cultured for seven days after forming (FIG. 1C). The cells originate from colorectal cancer (CRC) tissue.

FIGS. 2A to 2C illustrate Patient-Derived MOSs formed as described herein to include five dissociated primary tissue cells per MOS, cultured for one day after forming (FIG. 2A), cultured for three days after forming (FIG. 2B), and cultured for seven days after forming (FIG. 2C). The cells originate from colorectal cancer (CRC) tissue.

FIGS. 3A to 3C illustrate Patient-Derived MOSs formed as described herein to include twenty dissociated primary tissue cells per MOS, cultured for one day after forming (FIG. 3A), cultured for three days after forming (FIG. 3B), and cultured for seven days after forming (FIG. 3C). As in FIGS. 1A-1C and 2A-2C, the cells originate from colorectal cancer (CRC) tissue.

FIGS. 4A to 4E illustrate examples of Patient-Derived MOSs formed as described herein to include ten dissociated primary tissue cells per MOS. FIG. 4A shows the MOSs shortly after formation (at low magnification). FIG. 4B shows a higher magnification view of some of the MOSs of FIG. 4A taken after culturing for two days. FIG. 4C shows the MOSs after culturing for three days. FIG. 4D shows the MOSs after culturing for four days. FIG. 4E shows the MOSs after culturing for five days.

FIGS. 5A to 5B illustrate examples of MOSs formed as described herein from normal mouse liver hepatocytes, cultured for one day after forming (FIG. 1A), or cultured for ten days after forming (FIG. 1B). The mouse hepatocytes are taken from a normal (e.g., non-diseased) mouse liver.

FIG. 6 illustrates on the method of forming Patient-Derived MOSs from primary tissue (e.g., biopsy) samples, as described herein.

FIG. 7A schematically illustrates one example of an apparatus for forming Patient-Derived MOSs as described herein, including a microfluidic chip as part of the assembly. FIG. 7B is a perspective view of one example a microfluidics chip portion of an apparatus such as that shown in FIG. 7A. FIG. 7C schematically illustrates a portion of a microfluidics assembly for an apparatus for forming Patient-Derived MOSs, such as the one shown in FIG. 7A.

FIG. 8 shows one example of an image showing a plurality of Patient-Derived MOSs formed using an apparatus such as that shown in FIG. 7A, showing the Patient-Derived MOSs shortly after polymerizing, suspended within a channel containing the immiscible fluid (e.g., oil) prior to being aliquoted from the apparatus.

FIG. 9 is an image of a portion of a prototype microfluidics assembly for an apparatus for forming Patient-Derived MOSs, similar to that shown in FIG. 7C, illustrating the formation of Patient-Derived MOSs.

FIG. 10 illustrates a plurality of Patient-Derived MOSs as described herein, shortly after polymerization; the Patient-Derived MOSs are suspended in the immiscible fluid.

FIGS. 11A-11B illustrate another example of a plurality of Patient-Derived MOSs shortly after formation and suspended in the immiscible fluid (e.g., oil) at low magnification (FIG. 11A) and higher magnification (FIG. 11B).

FIGS. 12A-12B show a plurality of Patient-Derived MOSs following separation from the immiscible fluid within a few hours of formation of the Patient-Derived MOSs at low magnification (FIG. 12A) and higher magnification (FIG. 12B).

FIG. 13 is another example of an image showing a plurality of Patient-Derived MOSs formed as described herein.

FIG. 14 is a chart illustrating the size distribution of the diameters from a plurality of Patient-Derived MOSs formed from an exemplary biopsy sample.

FIGS. 15A-15B illustrate low and higher magnification views, respectively, of one example of a plurality of Patient-Derived MOSs formed from a dissociated tissue biopsy sample and a fluid matrix material, after polymerizing. FIG. 15A is an unstained image, while in FIG. 15B the MOSs have been stained with Trypan blue to show that the dissociated cells in the MOSs are alive.

FIGS. 16A-16B is another example, similar to that shown in FIGS. 15A-15B, showing low and higher magnification views, respectively, of one example of a plurality of Patient-Derived MOSs. FIG. 16A is an unstained image, while in FIG. 16B the MOSs have been stained with Trypan blue (arrows) to show that the dissociated cells in the MOSs indicated that the cell remail viable (e.g., living) within the MOS.

FIGS. 17A-17E illustrates one example of a method of assaying a plurality of Patient-Derived MOSs formed from a patient tumor biopsy, to determine a drug-response profile to multiple drug formulations. The illustrated procedure takes less than two weeks (e.g., approximately one week) from biopsy to results.

FIG. 18 schematically illustrates an example of a method for treating a patient including the formation and use of a plurality of Patient-Derived MOSs as part of the treatment procedure.

FIG. 19 schematically illustrates an example of a method for treating a patient including multiple iterations of rapidly forming and assaying a plurality of Patient-Derived MOSs as part of the treatment procedure.

FIG. 20 schematically illustrates a portion of an apparatus for forming a plurality of Patient-Derived MOSs as described herein.

FIG. 21 schematically illustrates a method of operating an apparatus for forming a plurality of Patient-Derived MOSs similar to that shown in FIG. 20.

FIGS. 22A-22D illustrate one example of a validation of a methods of using a plurality of Patient-Derived MOSs as described herein to identify drug resistance. FIG. 22A illustrates the use of traditional (“2D”) tumor cell assay methods for predicting drug resistance. FIG. 22B illustrates the use of one example of a Patient-Derived MOS method as described herein, to assay for drug resistance for predicting drug sensitivity. FIGS. 22C and 22D show that the Patient-Derived MOS based method accurately predicted the actual response of the tumor (drug responsive), unlike traditional cultured cells.

FIGS. 23A-23D illustrate another example validating the use of Patient-Derived MOSs as described herein to identify drug resistance, showing the predicted drug response to both Oxaliplatin and Irinotecan as consistent with actual tumor response following treatment with these drugs.

FIG. 24 illustrates one example of a drug screen using the Patient-Derived MOSs as described herein, in which a single tumor biopsy may generate a plurality of nearly-identical MOSs in large quantities extremely fast (e.g., within less than two weeks) and be quickly tested against a large number of drug formulations (e.g., 27 are shown) in parallel.

FIGS. 25A-25B illustrate examples of mouse liver MOSs formed from a mouse liver tissue, having diameters of 300 pm, and 1 cell per MOS. FIG. 25A shows the MOSs at day 1, and FIG. 25B shows the MOSs at day 10.

FIGS. 26A-26B illustrate examples of mouse liver MOSs formed from the partial hepatectomy mouse liver tissue, having diameters of 300 pm, and 25 cells per MOS similar to those shown in FIGS. 25A-25B. FIG. 26A shows the MOSs at day 1, and FIG. 26B shows the MOSs at day 10.

FIGS. 27A-27C illustrate examples of human liver MOSs formed from human liver tissue. FIG. 27A shows the MOSs at day 1, seeded with 40 cells/droplet. FIG. 27B and FIG. 27C show the MOSs at day 18. In FIG. 27B the MOSs are hepatocyte-like structures, while FIG. 27C shows Cholangiocyte-like MOSs.

FIGS. 28A-28D show examples of MOSs generated from a patient derived xenograft tumor line, having diameters of 300 pm, and 1 cell per MOS. FIG. 28A shows the MOSs at day 1, FIG. 28B shows the MOSs at day 3, FIG. 28C shows the MOSs at day 5 and FIG. 28D shows the MOSs at Day 7.

FIGS. 29A-29D show examples of MOSs generated from a patient-derived xenograft model, having diameters of 300 pm, and 5 cell per MOS. FIG. 29A shows the MOSs at day 1, FIG. 29B shows the MOSs at day 3, FIG. 29C shows the MOSs at day 5 and FIG. 29D shows the MOSs at Day 7.

FIG. 30 is a graph comparing the responses of traditional organoids and MOSs formed from a colorectal cancer patient-derived organoid to Oxalipalatin, showing a comparable response for the traditional organoids and MOSs.

FIG. 31 is a graph comparing the responses of traditional organoids and MOSs formed from two colorectal cancer patient-derived xenograft models to SN38 (7-Ethyl-10-hydroxy-camptothecin), showing comparable responses.

FIG. 32 is a graph comparing the responses of traditional organoids and MOSs formed from a colorectal cancer patient-derived xenograft model to 5-FU (Fluorouracil), showing comparable responses.

FIGS. 33A and 33B show examples of toxicity assays using mouse liver MOSs. FIG. 33A shows that the sizes of the tissue in the mouse liver MOSs in the control group are relatively large (as indicated by the arrows). In contrast, in FIG. 33 A, showing the acetaminophen (10 mM) treatment group, the tissue in most of the MOSs is smaller and contains many dead cells.

FIGS. 34A and 34B show examples of toxicity assays using human liver MOSs. FIG. 34A shows typical human liver MOSs observed in the control group including tissue structures (indicated by the arrows). FIG. 34B shows MOSs in an acetaminophen (10 mM) treatment group, showing atypical tissue structures (arrows) and debris.

FIGS. 35A and 35B respectively illustrate the effect of Nivolumab on immunooncology assays of MOSs based on pulmonary and renal tumors. FIG. 35A shows via Annexin V marker that Nivolumab induces apoptosis for a pulmonary tumor. FIG. 35B shows via Annexin V marker that Nivolumab induces apoptosis for a renal tumor.

FIGS. 36A and 36B illustrate the effect of Lenalidomine and Bortezoid on IO assays of MOSs based on a Multiple Myeloma (MM) biopsy. FIGS. 36A and 36B show via Caspase 3/7 dye that Lenalidomine induces apoptosis for a MM biopsy while Bortezoid does not.

FIG. 37 illustrates the effect of ESK1 on organoid death in a pulmonary tumor MOS.

FIG. 38 illustrates the effect of ESK1 with added PBMC on organoid death in a pulmonary tumor MOS.

FIG. 39 illustrates the effect of patient TILs on tumor cells in MOS.

FIG. 40 illustrates the effect of PBMC on tumor cells in MOS.

FIG. 41 illustrates the effect of combining the treatment of Nivolumab with TILS.

FIG. 42 illustrates the ability of patient T cells to penetrate conventional bulk organoids vs. MOS.

FIG. 43A illustrates a previous method for introducing CRISPR/Cas complex components into 3D tissue models. First a cell suspension is generated (i); the CRISPR/Cas complex components are introduced into single cells or spheroid structures for gene editing (ii); the edited cells are developed into a 3D matrix. FIG. 43B illustrates an example of the methods provided herein. MOS are generated (i); gene editing components are introduced into the MOS (ii); the cells in the MOS are genetically modified (iii). The difference between these methods is throughput: the first method would require 100 MOS generation steps for a 100 array library, whereas the method herein would enable the library to be tested after a single MOS generation step.

FIG. 44 illustrates how the methods provided herein enable arrayed library screening in 3D models. MOS are loaded into an array plate e.g., a multiwell plate (i); different guide RNA is added to each well and electroporated (ii); assays can be run with edited cells prepackaged into MOS (iii).

FIG. 45 shows the results of gene editing when gene editing components are introduced into a MOS using viral vector-based delivery methods.

FIG. 46 shows how electroporation methods were optimized to deliver gene editing components into a MOS. A total of 120 different electroporation conditions were tested, using eight different buffers, and 15 different pulse conditions.

FIG. 47 shows an individual MOS in which cells have been edited to express GFP after electroporation. The arrow denotes the outline of the MOS.

FIG. 48 shows the number of GFP positive cells after electroporation for four combinations of buffer and pulse conditions.

FIG. 49 illustrates gene editing of MOSs following delivery of CRISPR/Cas9 complex components. The difference between scramble and knockout is the editing efficiency in MOS.

FIG. 50 illustrates the process to select electroporation conditions for introduction of CRISPR/Cas9 into MOS. The X-axis represents different attempted optimization protocols. These included changes such as MOS diameter, Matrigel composition, incubation times and cell concentrations to develop a reliable protocol with high editing efficiency.

FIG. 51 shows the results of gene editing when gene editing components are delivered into a MOS using certain electroporation methods. This figure combines 3 different experiments by 3 different operators using the same protocol for gene editing in MOS. These results show that the method is consistent for high editing efficiencies in MOS.

FIG. 52 shows Cas9-GFP diffusion into MOS.

FIG. 53 shows FACS analysis of RNP MOS delivery.

### DETAILED DESCRIPTION

Provided herein are methods for delivering one or more components into a MicroOrganoSphere (MOS), the methods comprising introducing one or more components into the MOS by a delivery method.

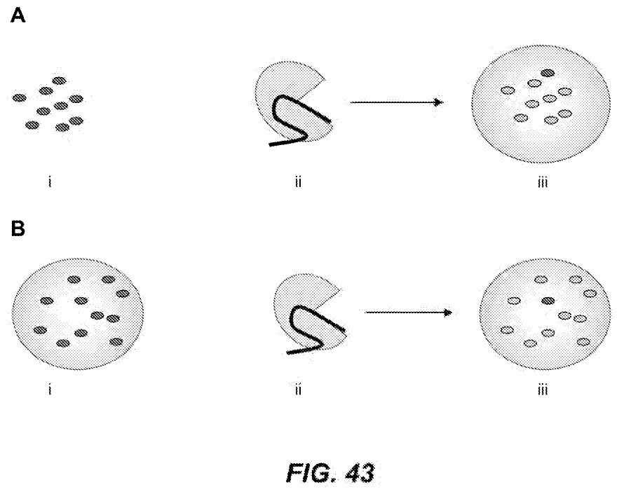

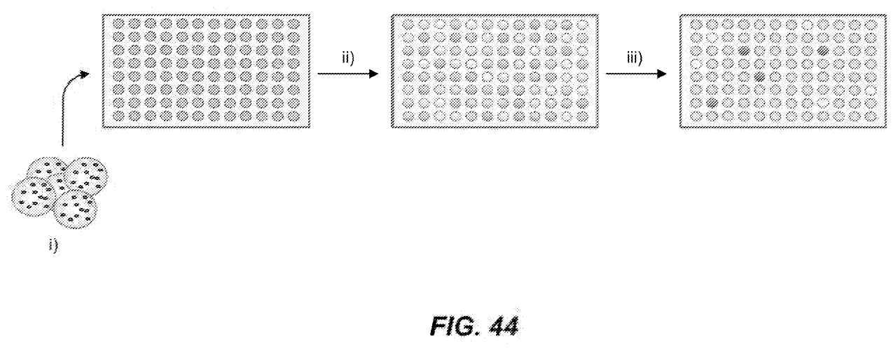

The methods relate to the delivery of components which are introduced following the formation of a MOS. An example of this is illustrated in FIG. 43B.

The one or more components may include any component to be introduced or tested in a MOS. For example, the one or more components may include proteins; peptides; polypeptides; DNA; RNA; siRNA; RNAI; plasmid DNA; viral particles; and antibodies or fragments thereof.

In particular, the one or more components are CRISPR/Cas complex components. In some embodiments the CRISPR/Cas complex components include a ribonucleoprotein which comprises guide RNA complexed with a Cas protein. In some embodiments, the components of the CRISPR/Cas9 complex may be encoded by DNA. In particular, the CRISPR/Cas complex components may be CRISPR/Cas9 complex components.

In some embodiments the one or more components are viral particles. The one or more components may comprise any viral particles. In some embodiments the viral particles may be derived from AAV, lentiviral, retroviral, SARS-COV, SARS-CoV-2, influenza, or other types of viruses.

Given the small size and large surface-to-volume ratio of a MOS, a MOS can be directly infected with viral particles. Merely by way of non-limiting examples, MOS can be directly infected with viral particles. In some embodiments the viral particles may be from AAV, lentiviral, retroviral, SARS-COV, SARS-COV-2, influenza, and other types of viruses. Advantageously, viral transduction efficiencies are much higher in a MOS culture as compared to conventional bulk organoid culture. The viral transduction efficiencies could be adjusted by changing the MOI (e.g., 0.1-50 MOI) of viruses used in the infection. There are a number of ways of achieving infection of MOS. By way of example, for infection, the MOS droplets can be spun down at 200 g for 3 min, after removing the supernatant, 200 μL of virus containing buffer can be added to the MOS pellets and resuspended followed by an incubation at 37° C. for 2-4 h. In one embodiment, 0.4% BSA 1×PBS with Ca+ and Mg+ can be used. In other embodiments, media with no serum can be used. After incubation, the viral containing supernatants can be removed and replaced with the complete media. After 24 h to 72 h of infection, the efficiency of viral infection can be monitored by fluorescent imaging and other approaches (e.g., PCR, western blot, antibiotic selection).

As MOS can be directly infected with viral particles very efficiently, there are numerous potential applications of using a MOS-based viral delivery system. Merely by way of non-limiting examples, introducing viral particles into MOS can be used for host and pathogen interaction studies; anti-viral drug screening; any viral delivery-based genome editing approaches (e.g., CRISPR/Cas9, transgene, gene knock-down); and creating reporter lines for drug screening.

In some embodiments the MOS comprises one or more cells. A MOS may comprise any cell type or combination thereof. The cell types may be selected from any tissue origin. The cell types may be selected from but are not limited to cancerous cells (from a solid tumor or cancer of the blood or bone marrow); non-cancerous cells from any tissue origin, immune cells, stromal cells, hepatocytes; respiratory tract cells; lung cancer cells, colorectal cancer cells, melanoma cells, and engineered cells (e.g. CAR T cells).

As used herein, a tumorsphere is a collection of tumor like patient derived live cells recapitulating the in vivo tumor environment.

As used herein, respiratory tract cells include, but are not limited to, sinonasal mucosa, trachea, proximal lung, and distal lung cells.

Suitable delivery methods for delivering components into a MOS may be selected from methods of electroporation; lipid-based delivery methods; and viral vector-based delivery methods. Viral vector-based delivery methods include lentiviral, AAV and retroviral-based vectors.

Methods for electroporation may be performed using any means or apparatus suitable for electroporation. Methods for electroporation may be performed using standard laboratory equipment including any electroporation system or instrument. In some embodiments, electroporation methods utilize a Lonza Nucleofector 4D system. In some embodiments, the electroporation methods utilize a Lonza buffer. In some embodiments the Lonza buffer is P1. In some embodiments the Lonza buffer is P3. Lonza buffer P3 consists of: 5 mM KCl, 15 mM MgCl2; 15 mM HEPES; 150 mM Na2HPO4/NaH2PO4 pH7.2; 50 mM Sodium Succinate. In some embodiments the electroporation method utilizes specific pulse conditions. In some embodiments the pulse conditions are selected from one of the following Lonza programs: CA-137. CM-138, CM-137, CM-150, DN-100, DS-138, DS-137, DS-130, DS-150, DS-120, EH-100, EO-100, EN-138, EN-150, and EW-113. In some embodiments the pulse conditions are selected from Lonza programs CA-137; DS-150; CM-137; and EN-138. In one embodiment the buffer is P1 and pulse conditions are CA-137. In another embodiment the buffer is P3 and the pulse conditions are EN-138.

The one or more components may be incubated with the MOS prior to electroporation. The one or more components may be incubated with the MOS for less than 1 minutes to 60 minutes prior to electroporation. In some embodiments the one or more components may be incubated with the MOS for 10 minutes prior to electroporation. In some embodiments the one or more components may be incubated with the MOS for 20 minutes prior to electroporation. In some embodiments no incubation is necessary.

The one or more components may be incubated with the MOS at a temperature of 20 to 40 degrees centigrade. In some embodiments the one or more components may be incubated with the MOS at a temperature of 37 degrees centigrade.

In some embodiments the MOSs are dispensed into a multiwell plate. In some embodiments each well of the multiwall plate contains 20 MOS droplets. In some embodiments each well of the multiwell plate contains 40-80 MOS droplets. In some embodiments each well of the multiwell plate contains 100-200 MOS droplets. In some embodiments, the number of MOS droplets in each well will depend on the size of the multiwell plate, a larger multiwell plate with more wells will contain fewer MOS droplets in each well. For example, a 384 well plate may contain 40-80 MOS droplets per well, a 96 well plate may contain 100-200 MOS droplets per well, a 24 well plate may contain 1000 MOS droplets per well. In some embodiments the method is high-throughput, allowing an array of MOSs and conditions to be investigated in a single assay. The methods herein maintain the cell viability of the one or more cells within the MOS such that a 3D microenvironment develops and/or the MOS and tumorspheres therein develop into organoid and/or tissue models.

Provided herein are methods of editing DNA or RNA comprised within a MOS, the methods comprising: a) delivering one or more CRISPR/Cas complex components into the MOS by a delivery method; and b) incubating the MOS under conditions suitable for CRISPR/Cas mediated DNA or RNA editing. In some embodiments the DNA or RNA editing efficiency is from 25% to 99%, or from 50% to 99%, about 80%, greater than 80%, or greater than 90%.

The CRISPR/Cas complex components may be delivered by any method. In some embodiments the CRISPR/Cas complex components are delivered by a method described herein. In some embodiments the CRISPR/Cas complex components are delivered by a delivery method comprising electroporation. In some embodiments the electroporation method is an electroporation method described herein.

Provided herein are methods of drug screening comprising: a) delivering into a MOS a drug and one or more additional components; and b) assessing the efficacy of the drug. In some embodiments, one or more additional components are introduced into a MOS prior to delivering a drug. In some embodiments, the drug is delivered into a MOS prior to delivery or one or more additional components.

In some embodiments the one or more additional components are DNA or RNA editing components. In some embodiments, the DNA or RNA editing components are CRISPR/Cas complex components. In some embodiments, DNA or RNA comprised within the MOS is edited by CRISPR/Cas, and the efficacy of the drug in MOS comprising edited DNA or RNA is compared to the efficacy of the drug in MOS comprising unedited DNA or RNA.

Provided herein is a MOS produced by the methods provided herein.

Provided herein is a MOS comprising one or more CRISPR/Cas9 complex components.

There are numerous applications of the MOS gene editing technologies described herein. Merely by way of example, this method can be used for high-throughput testing of cells in MOS where each well is edited in a specific way. Some of the potential uses for this technique are testing for mutation specific effects of drugs, the mechanisms of action of drugs in a 3D tissue system, and defining which genes are desirable to target with a pharmaceutical for a specific patient or patient population. In particular, high-throughput methods can be used to introduce genetic alterations to screen for proteins that dampen the immune response to cancer for several patients with the aim of identifying new drug targets. Although this technology can be applied to discover or develop any type of drug, it is especially helpful in determining and evaluating drug targets for immunooncology drugs by testing the effects of various mutations.

### DETAILED DESCRIPTION OF MOS FORMATION AND APPLICATIONS

In general, described herein are MOSs, methods and apparatuses for forming them, and methods and apparatuses for using them, e.g., to assay for tissue (including, but not limited to cancerous tissue) responses.

The MOSs described herein are typically spheres formed from dissociated primary cells distributed within the base material. These MOSs may have a diameter of between about 50 μm and about 500 μm (e.g., between about 50 μm and about 400 μm, about 50 μm and about 300 μm, about 50 μm and about 250 μm, etc.), and may initially contain between about 1 and 1000 dissociated primary cells distributed within the base material (e.g., between about 1 and 750, between about 1 and 500, between about 1 and 400, between about 1 and 300, between about 1 and 200, between about 1 and 150, between about 1 and 100, between about 1 and 75, between about 1 and 50, between about 1 and 40, between about 1 and 30, between about 1 and 20, etc.).

Surprisingly, despite their small size (often between about 50-250 μm), and low cell density (e.g., often less than 100 cells per MOS), these MOSs may be used immediately or cultured for a very brief period of time (e.g., 14 days or less, 10 days or less, 7 days or less, 5 days or less, etc.) and may allow the cells within the MOSs to survive while maintaining much, if not all, of the characteristics of the tissue, including tumor tissue, from which they were extracted. The survival rate of the cells within the MOSs is remarkably high, and the MOSs may be cultured for days (or weeks) through multiple passages, in which the cells will divide, cluster and form structures similar to the parent tissue. Also surprisingly, the cells from the dissociated tissue within the MOS can form morphological structures inside even the smallest MOSs; although in some applications, the presence of such structures is not necessary for the utility of these MOSs (e.g., they may be used before substantial structural reorganization has occurred) they may be particularly useful.

The methods and apparatuses described herein for forming and using MOSs may be used to create many (e.g., greater than 10,000) MOSs from a single biopsy. These MOSs may be used screen for drug compositions that may predict what therapies may be effectively applied to the patient from whom the biopsy was taken. This may be useful, for example, in toxicity screen for drugs or other chemical compositions, from healthy normal tissue and/or from cancerous (e.g., tumor) tissue. In particular, the MOSs, methods and apparatuses for forming them and methods and apparatuses for testing them may be used for screening to identify one or more drug compositions or combinations of drug compositions that may effectively treat the patient (e.g., a cancer patient) prior to undergoing the drug therapy. This may allow, for example, very rapid screening of a cancer patient before they would otherwise undergo months of chemotherapy that may not be effective for them.

Thus, described herein are high-throughput drug screening methods (and apparatuses for performing these methods) using a single patient-specific biopsy (or other appropriate tissue/cell source). Described herein are droplet formed MOSs that may be formed from patient-derived tumor samples that have been dissociated and suspended in a basement matrix (e.g., MATRIGEL). The MOSs can be patterned onto a microfluidic microwell array, to be incubated, and dosed with drug compounds. This miniaturized assay maximizes the use of tumor samples, and enables more drug compounds to be screened from a core biopsy at much lower cost per sample.

Patient-derived models of cancer (PDMC), such as cell lines, organoids and patient-derived xenografts (PDXs) are increasingly being accepted as “standard” preclinical models to facilitate the identification and development of new therapeutics. For example, large-scale drug screens of cell lines and organoids derived from cancer patients have been used to identify sensitivity to a large number of potential therapeutics. PDXs are also used to predict drug response and identify novel drug combinations. Although precision medicine strategies are in development through the exploration of these various PDMC models, there are substantial barriers to their effective use. For example, patient derived organoids (PDO) are believed to be the most accurate in depicting patient tumors, as studies have shown that phenotypic and genotypic profiling of organoids often show a high degree of similarity to the original patient tumors. Unfortunately, at least two limitations hinder the use of PDO to guide therapy. Firstly, it takes several months to develop and test drug sensitivity in organoids, which decreases the clinical applicability. Secondly the number of organoids obtained from a clinically relevant 18-gauge core biopsy is not sufficient to perform high throughput drug screen. Ideally, an assay should be performed from a single core biopsy within 7-10 days. The MOSs and methods of making and using them described herein may address these clinical limitations.

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by one of ordinary skill in the art, definitions are set forth herein to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

The term “an unpolymerized mixture” is used herein to refer to a composition comprising biologically-relevant materials, including a dissociated tissue sample and a first fluid matrix material. The fluid matrix material is typically a material that may be polymerized to form a support or support network for the dissociated tissue (cells) dispersed within it. Once polymerized, the polymerized material may form a hydrogel and may be formed or and/or may include proteins forming the biocompatible medium, in addition to the cells. A suitable biocompatible medium for use in accordance with the presently-disclosed subject matter can typically be formed from any biocompatible material that is a gel, a semi-solid, or a liquid, such as a low-viscosity liquid, at room temperature (e.g., 25° C.) and can be used as a three-dimensional substrate for cells, tissues, proteins, and other biological materials of interest. Exemplary materials that can be used to form a biocompatible medium in accordance with the presently-disclosed subject matter include, but are not limited to, polymers and hydrogels comprising collagen, fibrin, chitosan, MATRIGEL™ (BD Biosciences, San Jose, Calif.), polyethylene glycol, dextrans including chemically crosslinkable or photo-crosslinkable dextrans, and the like, as well as electrospun biological, synthetic, or biological-synthetic blends. The biocompatible medium may be comprised of a hydrogel.

The term “hydrogel” is used herein to refer to two- or multi-component gels comprising a three-dimensional network of polymer chains, where water acts as the dispersion medium and fills the space between the polymer chains. Hydrogels used in accordance with the presently-disclosed subject matter are generally chosen for a particular application based on the intended use of the structure, taking into account the parameters that are to be used to form the MOSs, as well as the effect the selected hydrogel will have on the behavior and activity of the biological materials (e.g., cells) incorporated into the biological suspensions that are to be placed in the structure. Exemplary hydrogels of the presently-disclosed subject matter can be comprised of polymeric materials including, but not limited to: alginate, collagen (including collagen types I and VI), elastin, keratin, fibronectin, proteoglycans, glycoproteins, polylactide, polyethylene glycol, polycaprolactone, polycolide, polydioxanone, polyacrylates, polyurethanes, polysulfones, peptide sequences, proteins and derivatives, oligopeptides, gelatin, elastin, fibrin, laminin, polymethacrylates, polyacetates, polyesters, polyamides, polycarbonates, polyanhydrides, polyamino acids carbohydrates, polysaccharides and modified polysaccharides, and derivatives and copolymers thereof as well as inorganic materials such as glass such as bioactive glass, ceramic, silica, alumina, calcite, hydroxyapatite, calcium phosphate, bone, and combinations of all of the foregoing.

With further regard to the hydrogels used to produce the MOSs described herein, the hydrogel may be comprised of a material selected from the group consisting of agarose, alginate, collagen type I, a polyoxyethylene-polyoxypropylene block copolymer (e.g., Pluronic® F127 (BASF Corporation, Mount Olive, N.J.)), silicone, polysaccharide, polyethylene glycol, and polyurethane. The hydrogel may be comprised of alginate.

The MOSs described herein may also include biologically-relevant materials. The phrase “biologically-relevant materials” may describe materials that are capable of being included in a biocompatible medium as defined herein and subsequently interacting with and/or influencing biological systems. For example, in some implementations, the biologically-relevant materials are magnetic beads (i.e., beads that are magnetic themselves or that contain a material that responds to a magnetic field, such as iron particles) that can be combined as part of the unpolymerized material to produce MOSs that can be used in the methods and compositions (e.g., for the separation and purification of MOSs). As another example, in other implementations, the biologically-relevant materials may include additional cells, in addition to the dissociated tissue sample (e.g., biopsy) material. In the unpolymerized mixture the dissociated tissue sample and the additional biologically relevant material can exist in a uniform mixture or as a distributed mixture (e.g., on just one half or other portion of the MOS, including just in the core or just in the outer region of the formed MOSs). The additional biologically-relevant material within the unpolymerized material may be suspended with the dissociated tissue sample in suspension, e.g., prior to polymerization of the droplet forming the MOS.

The biologically relevant material that may be included with the dissociated tissue sample (e.g., biopsy) material may contain a number of cell types, including preadipocytes, mesenchymal stem cells (MSCs), endothelial progenitor cells, T cells, B cells, mast cells, and adipose tissue macrophages, as well as small blood vessels or microvascular fragments found within the stromal vascular fraction.

In general, with respect to the dissociated tissue sample, e.g., biopsy, material that is included in the MOSs described herein, these tissues may be any appropriate tissue from a patient, typically taken by biopsy. Although non-biopsy tissue may be used, in general, these tissues (and the resulting dissociated cells) may be primary cells taken from a patient biopsy as described above, e.g., by a needle biopsy. Tissues may be from a healthy tissue biopsy or from cancerous (e.g., tumor) cell biopsy. The dissociated cells may be incorporated into a MOS of the presently-disclosed subject matter, based on the intended use of that MOS. For example, relevant tissues (e.g., dissociated biopsy tissue) may typically include cells that are commonly found in that tissue or organ (or tumor, etc.). In that regard, exemplary relevant cells that can be incorporated into MOSs of the presently-disclosed subject matter include neurons, cardiomyocytes, myocytes, chondrocytes, pancreatic acinar cells, islets of Langerhans, osteocytes, hepatocytes, Kupffer cells, fibroblasts, myoblasts, satellite cells, endothelial cells, adipocytes, preadipocytes, biliary epithelial cells, and the like. These types of tissues may be dissociated by conventional techniques known in the art. Suitable biopsied tissue can be derived from: bone marrow, skin, cartilage, tendon, bone, muscle (including cardiac muscle), blood vessels, corneal, neural, brain, gastrointestinal, renal, liver, pancreatic (including islet cells), lung, pituitary, thyroid, adrenal, lymphatic, salivary, ovarian, testicular, cervical, bladder, endometrial, prostate, vulval and esophageal tissue. Normal or diseased (e.g., cancerous) tissue may be used. The tissue may arise from tumor tissue, including tumors originating in any of these normal tissues.

Once formed the MOSs may be cryopreserved and/or cultured. Cultured MOSs may be maintained in suspension, either static (e.g., in a well, vial, etc.) or in motion (e.g., rolling or agitated). The MOSs may be cultured using known culturing techniques. Exemplary techniques can be found in, among other places; Freshney, Culture of Animal Cells, A Manual of Basic Techniques, 4th ed., Wiley Liss, John Wiley & Sons, 2000; Basic Cell Culture: A Practical Approach, Davis, ed., Oxford University Press, 2002; Animal Cell Culture: A Practical Approach, Masters, ed., 2000; and U.S. Pat. Nos. 5,516,681 and 5,559,022.

The MOSs may be formed by forming a droplet of the unpolymerized mixture (e.g., a chilled mixture) of a dissociated tissue sample and a fluid matrix material in an immiscible material, such as a fluid hydrophobic material (e.g., oil). For example, a MOS may be formed by combining a stream of unpolymerized material with one or more streams of the immiscible material to form a droplet. The density of the cells present in the droplet may be determined by the dilution of the dissociated material (e.g., cells) in the unpolymerized material. The size of the MOSs may correlate to the size of the droplet formed. In general, the MOS is a spherical structure having a stable geometry.

The practice of the presently disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, M. J. Gait, ed., 1984; Nucleic Acid Hybridization, 15 D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor 20 Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

As used herein a drug composition may include any drug, drug dilution, drug formulation, compositions including multiple drugs (e.g., multiple active ingredients), drug formulations, drug forms, drug concentrations, combination therapies, and the like. A drug formulation may refer to a formulation comprising a mixture of a drug and one or more inactive ingredients. As used herein the term “passaged” may refer to the average number of doublings of the cells within the MOSs. Although traditional passage number refers to the transfer or subculture of cells from one culture vessel to another, the cells within a MOS may be stably retained within the same MOS, and may continue to grow and divide. Thus, the passage number as used herein typically refers to the average number of doublings undergone by the dissociated cells from the biopsied tissue within the MOSs. The population doubling number is the approximate number of doublings that the cell population has undergone since isolation (e.g., since forming of the MOSs from the freshly dissociated biopsy tissue). In general, the MOSs described herein may be cultured for a short period of time relative to the growth, e.g., doublings, of some or all of the cells within the MOSs (e.g., fewer than 10 passages, fewer than 9 passages, fewer than 8 passages, fewer than 7 passages, fewer than 6 passages, fewer than 5 passages, fewer than 4 passages, fewer than 3 passages, etc.).

During culturing, the cells from the dissociated, biopsied tissue in the MOSs can aggregate, cluster, or assemble within the MOSs. Aggregates of cells may be highly organized, and may form defined morphology or may be a mass of cells that have clustered or adhered together. The organization may reflect the tissue of origin. Although the MOSs may contain a single cell type (homotypic), more typically these MOSs may contain more than one cell type (heterotypic).

As mentioned, the (e.g., biopsy) tissue used to form the MOSs (e.g., the dissociated tissue) may be derived from a normal or healthy biological tissue, or from a biological tissue afflicted with a disease or illness, such as a tissue or fluid derived from a tumor. The tissue used in the MOSs may include cells of the immune system, such as T lymphocytes, B lymphocytes, polymorphonuclear leukocytes, macrophages and dendritic cells. The cells may be stem cells, progenitor cells or somatic cells. As described in further detail below, the presence of these immune cells can be used to enhance the efficacy and accuracy of drug/biologic testing. The tissue may be mammalian cells such as human cells or cells from animals such as mice, rats, rabbits, and the like.

In general, the tissue (and resulting cells) may generally be taken from a biopsy to form the MOSs. Thus, the tissue may be derived from any of a biopsy, a surgical specimen, an aspiration, a drainage, or a cell-containing fluid. Suitable cell-containing fluids include any of blood, lymph, sebaceous fluid, urine, cerebrospinal fluid or peritoneal fluid. For example, in patients with transcoelomic metastasis, ovarian or colon cancer cells may be isolated from peritoneal fluid. Similarly, in patients with cervical cancer, cervical cancer cells may be taken from the cervix, for example by large excision of the transformation zone or by cone biopsy. Typically, such MOSs will contain multiple cell types that are resident in the tissue or fluid of origin. The cells may be obtained directly from the subject without intermediate steps of subculture, or they may first undergo an intermediate culturing step to produce a primary culture. Methods for harvesting cells from biological tissue and/or cell containing fluids are well known in the art. For example, techniques used to obtain cells from biological tissue include those described by R. Mahesparan (Extracellular matrix-induced cell migration from glioblastoma biopsy specimens in vitro. Acta Neuropathol (1999) 97:231-239).

Generally, the cells are first dissociated or separated from each other before forming the MOSs. Dissociation of cells may be accomplished by any conventional means known in the art. Preferably, the cells are treated mechanically and/or chemically, such as by treatment with enzymes. By ‘mechanically’ we include the meaning of disrupting connections between associated cells, for example, using a scalpel or scissors or by using a machine such as an homogenizer. By ‘enzymatically’ we include the meaning of treating the cells with one or more enzymes that disrupt connections between associated cells, including for example any of collagenase, dispases, DNAse and/or hyaluronidase. One or more enzymes may be used under different reaction conditions, such as incubation at 37° C. in a water bath or at room temperature.

The dissociated tissue may be treated to remove dead and/or dying cells and/or cell debris. The removal of such dead and/or dying cells may be accomplished by any conventional means known to those skilled in the art, for example using beads and/or antibody methods. It is known, for example, that phosphatidylserine is redistributed from the inner to outer plasma membrane leaflet in apoptotic or dead cells. The use of Annexin V-Biotin binding followed by binding of the biotin to streptavidin magnetic beads enables the separation of apoptotic cells from living cells. Similarly, removal of cell debris may be achieved by any suitable technique in the art, including, for example, filtration.

The dissociated cells may be suspended in a carrier material prior to combining with the fluid matrix material, and/or the fluid matrix material may be referred to as a carrier material. The carrier material may be a material that has a viscosity level that delays sedimentation of cells in a cell suspension prior to polymerization and formation of the MOSs. A carrier material may have sufficient viscosity to allow the dissociated biopsy tissue cells to remain suspended in the suspension until polymerization. The viscosity required to achieve this can be optimized by the skilled person by monitoring the sedimentation rate at various viscosities and selecting a viscosity that gives an appropriate sedimentation rate for the expected time delay between loading the cell suspension into the apparatus forming the MOSs by polymerizing the droplets of the unpolymerized material including the cells. The unpolymerized material may be flowed or agitated by the apparatus even where lower viscosity materials are used, in order to keep the cells in suspension and/or distributed as desired.

As mentioned above, the unpolymerized mixture, including the dissociated tissue sample and the fluid matrix material, may include one or more components, e.g., biologically-relevant materials. For example, a biologically-relevant material that may be included may be any of: an extracellular matrix protein (e.g. fibronectin), a drug (e.g. small molecules), a peptide, or an antibody (e.g., to modulate any of cell survival, proliferation or differentiation); and/or an inhibitor of a particular cellular function. Such biologically-relevant materials may be used, for example, to increase cell viability by reducing cell death and/or activation of cell growth/replication or to otherwise mimic the in vivo environment. The biologically-relevant materials may include or may mimic one or more of the following components: serum, interleukins, chemokines, growth factors, glucose, physiological salts, amino acids and hormones. For example, the biologically-relevant materials may supplement one or more agents in the fluid matrix material. The fluid matrix material may be a synthetic gel (hydrogel) and may be supplemented by one or more biologically-relevant materials. The fluid matrix may be a natural gel. Thus, the gel may be comprised of one or more extracellular matrix components such as any of collagen, fibrinogen, laminin, fibronectin, vitronectin, hyaluronic acid, fibrin, alginate, agarose and chitosan. For example, MATRIGEL comprises bioactive polymers that are important for cell viability, proliferation, development and migration. For example, the matrix material may be a gel that comprises collagen type 1 such as collagen type 1 obtained from rat tails. The gel may be a pure collagen type 1 gel or may be one that contains collagen type 1 in addition to other components, such as other extracellular matrix proteins. A synthetic gel may refer to a gel that does not naturally occur in nature. Examples of synthetic gels include gels derived from any of polyethylene glycol (PEG), polyhydroxyethyl methacrylate (PHEMA), polyvinyl alcohol (PVA), polyethylene oxide (PEO).

### MOSs

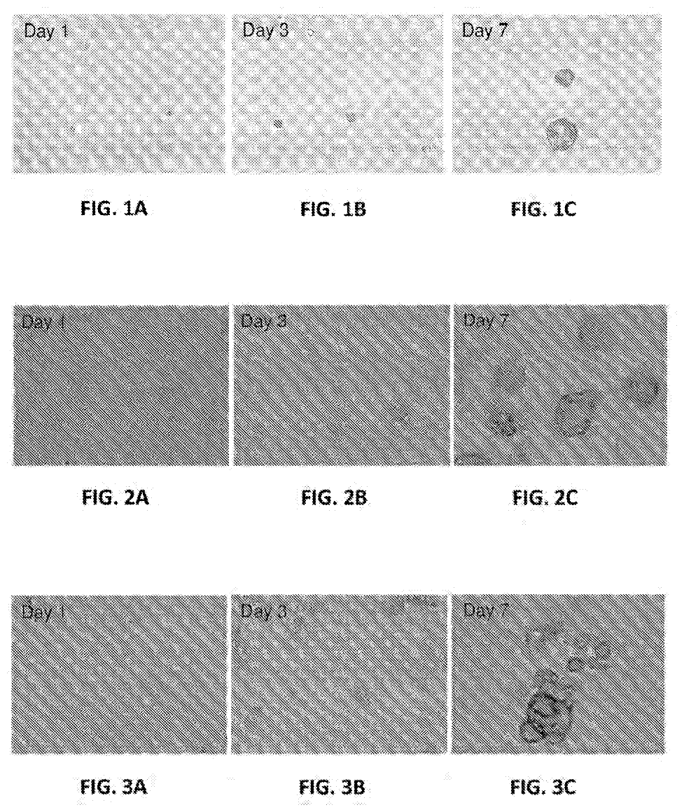

Examples of MOSs are shown in FIGS. 1A-1C, 2A-2C, 3A-3C and 4A-4E. For example, FIGS. 1A-1C illustrate MOSs formed having a single cell per MOS. As shown, the MOSs are all approximately the same size, e.g., approximately 300 μm diameter. FIG. 1B shows MOSs formed at the same time after 3 days in culture. The cells have expanded in size, in some cases doubling and/or growing. By seven days in culture, as shown in FIG. 1C, the cells have doubled multiple times, showing clusters or masses of cells.

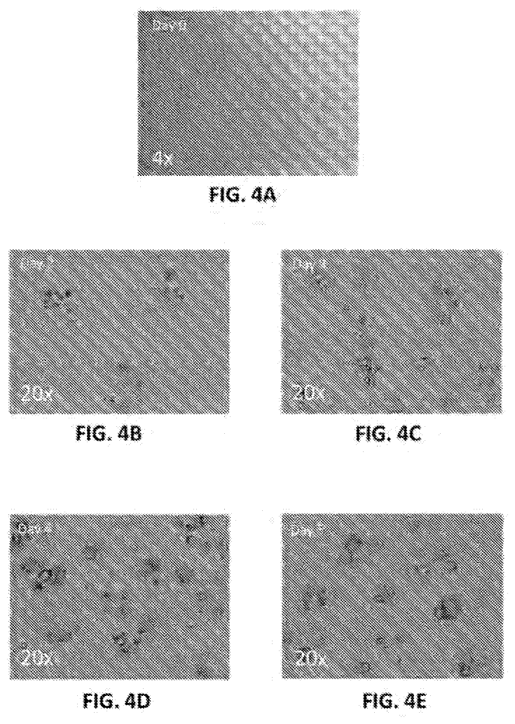

Similar results are shown in FIGS. 2A-2C and 3A-3C showing MOSs formed from five cells per MOS or 20 cells per MOS, respectively. In FIGS. 4A-4E, the MOSs are shown immediately after formation, and cultured for five days, in which nearly-identical MOSs (e.g., having the same diameter) each include 10 cells per MOS. In FIG. 4A, the MOSs are shown immediately after forming, still surrounded by the immiscible fluid, in this case, oil, at day 0. The MOSs are removed from the immiscible fluid and washed, and cultured for five days. FIG. 4B shows the MOSs after 2 days, FIG. 4C shows the MOSs after 3 days, and FIGS. 4D and 4E show the MOSs at 4 and 5 days, respectively. FIGS. 4A-4E show that the dissociated tissue (cells) from the biopsy within the MOSs are viable and growing within nearly all of the MOSs at comparable rates. As will be described in greater detail there, these MOSs may be formed in large amounts from even a single, average-sized biopsy and may result in many hundreds or thousands (e.g., 500, 750, 1000, 2000, 5000, 10,000 or more) of MOSs that include a significant number of viable cells, allowing multiple rapid assays to be performed in parallel.

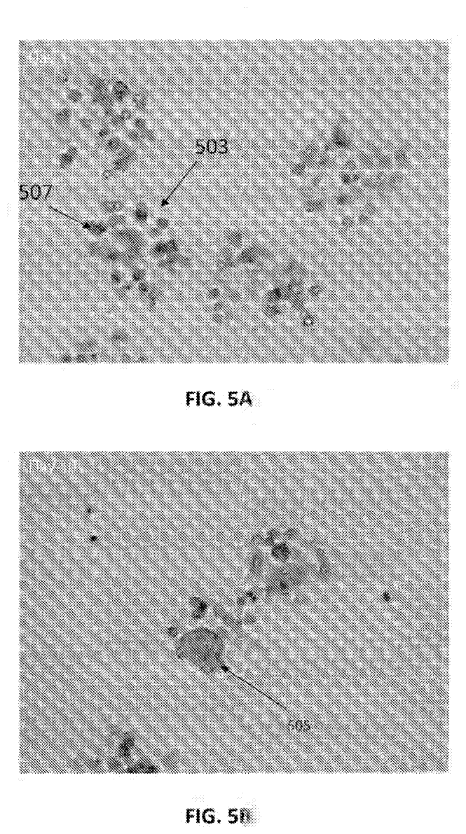

FIGS. 5A and 5B illustrate an example of MOSs formed as described herein from a dissociated biopsy of mouse liver, e.g., showing mouse hepatocytes distributed within a polymerized fluid matrix material (in this example, MATRIGEL). Each MOS includes the polymerized matrix material 503 formed into a sphere having a diameter, e.g., of about 300 μm, in which a set number of hepatocytes 507 are dispersed. In FIG. 5A, the MOSs are shown one day after biopsying, dissociation and forming of the MOSs. These MOSs were then cultured for 10 days, during which time the cells (hepatocytes) remained viable and grew, in many cases doubling multiple times to form structures 505, as shown in FIG. 5B.

The MOSs may generally include the dissociated (e.g., biopsy) tissue (e.g., cells) in a fixed or known number of cells and/or concentration (cells/ml or cells/mm3) within the MOSs. As mentioned above, this matrix material may be natural polymers, such as one or more of: alginate, agarose, hyaluronic acid, collagen, gelatin, fibrin, elastin; or a synthetic polymer, such as one or more of: polyethylene glycol (PEG) and polyacrylamide. Both organic and inorganic synthetic polymers may be used.

The number of cells initially included in the MOSs may be selected from between 1 cell up to several hundred. In particular, in some assays (e.g., drug toxicity assays) it may be beneficial to include between about 1-75 or between about 1-50 (e.g., lower numbers of cells). The number of cells per MOS may be set or selected by the user. As described below, the apparatus may include one or more controls to set the number of cells from the primary tissue to include in each MOS. The number of cells may be chosen or set based on how the user intends to use the MOSs. For example, MOSs having very low number of cells (e.g., 1 cell per MOS, 1-5 cells per MOS, etc.) may be particularly suitable for studying clonal diversity (e.g., for tumor heterogeneity). Since each MOS grows from a single cell, we can observe which clones are drug resistant and these specific MOSs may be examined (e.g., by genomic sequencing) to determine the genomic (mutation) diversity related to the particular clone. A low to moderate number of cells per MOS (e.g., between about 3-30 cells, 5-30 cells, 5-25 cells, 5-20 cells, 10-25 cells, etc.) may be particularly useful for rapid drug testing, including toxicity testing as these MOSs typically grow quickly. A larger number of cells per MOS (e.g., between about 20-100 cells, e.g., 30-100 cells, 40-100 cells, greater than 50 cells, etc.) may be particularly suitable for mimicking tissue composition in each MOS, as the MOSs may contain different lineages, potentially including epithelial (or cancer, etc.) and mesenchymal (or stromal, immune, blood vessel, etc.) cells.

The MOSs may be formed in any appropriate size, which may be matched to the number of cells to be included. For example, the size may be as small as about 20 μm, up to 500 μm in diameter (e.g., 50 or 100 μm on average, e.g., between about 100-200 μm, etc.). The size may be about 300 μm in which between about 10-50 cells (e.g., between about 10-30 cells) are included in each MOS. The number of cells and the size may be varied and/or may be controlled. The number of cells and/or the size of the MOSs may be set by one or more controls on the apparatus forming the MOSs. For example, the size of the MOSs and/or the density of cells within the MOSs may be adjusted by adjusting the flow rates and/or the concentration of the dissociated tissue sample (e.g., the cells from a biopsy).

As shown in FIGS. 1A-5B, even after culturing the MOSs described herein allow for viable and healthy cells through the entire volume of the MOS. The size of the MOSs and/or the number of cells to be included in the MOSs may be selected based on how the MOSs are expected or intended to be used. For example, if the MOSs are to be used to examine relationships between cells of the biopsied material, the MOSs may be formed having multiple cells and may be cultured for extended periods of time (e.g., up to one week or more).

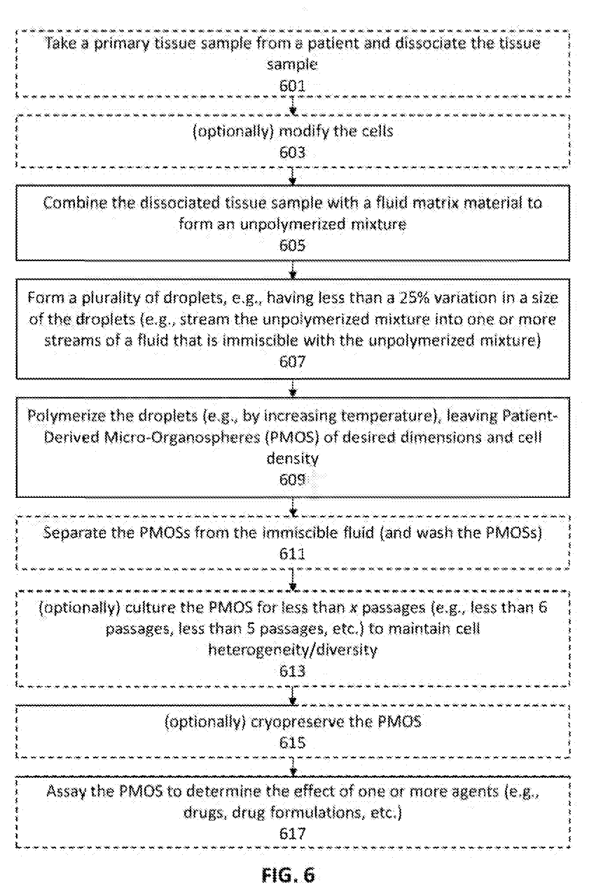

The MOSs described herein may be made by combining a dissociated tissue sample, e.g., a biopsy sample, with a fluid matrix that may be polymerized in a controlled manner to form the MOSs. FIG. 6 illustrates one method of forming MOSs. Optionally, the method may include taking the sample from a patient, such as taking a biopsy from a patient tissue 601. As mentioned above, the biopsy may be taken, e.g., using a biopsy needle or punch. For example, the biopsy may be taken with a 14-gauge, a 16-gauge, an 18-gauge, etc. needle that is inserted into the patient tissue to remove the biopsy. After removing the tissue from the patient, the tissue may be processed to dissociate the material, either mechanically and/or chemically. The dissociated cells may be immediately used to form the MOSs, as described; all or some of the cells may be modified, such as by genetically modifying the cells 603, for example, by transfection, electroporation, etc.

The dissociated tissue sample from the biopsy material may be combined with the fluid (e.g., liquid) matrix material to form the unpolymerized mixture 605. This unpolymerized mixture may be held in an unpolymerized state, so that the cells from the dissociated tissue may remain suspended within the mixture. The cell may remain suspended and unpolymerized by keeping them chilled, e.g., at room temperature of below (e.g., between 1-25 degrees C.).

The unpolymerized mixture may then be dispensed as droplets, e.g., into an immiscible material, such as an oil, in a manner that controls the formation of the size of the droplets and therefore the size of the MOSs formed 607. For example, uniformly-sized droplets may be formed by combining a stream of the unpolymerized material into one or more (e.g., two converging) streams of the immiscible material (e.g., oil) so that the flow rates and/or pressures of the two streams may determine how droplets of the unpolymerized material are formed as they intersect the immiscible material. The droplets may be polymerized 609 to form the MOSs in the immiscible material. The immiscible material may be heated or warmed to a temperature that causes the unpolymerized mixture (e.g., the fluid matrix material in the unpolymerized material) to polymerize. Once formed, the MOSs may be separated from the immiscible fluid, e.g., the MOSs may be washed to remove the immiscible fluid 611, and placed in a culture media to allow the cells within the MOSs to grow. The MOSs may be cultured for any desired time, or may be cryopreserved and/or assayed immediately. The MOSs may be cultured for a brief period of time (e.g., for between 1-3 days, between 1-4 days, between 1-5 days, between 1-6 days, between 1-7 days, between 1-8 days, between 1-9 days, between 1-10 days, between 1-11 days, between 1-14 days etc.). This may allow the cells derived from the dissociated biopsy tissue to grow and/or divide (e.g., double) for up to five or six passages. After culturing, the cells may be either or both cryopreserved 615 and/or assayed 617. Examples of assays that may be used are also described herein.

In any of these methods and apparatuses described herein, the MOSs may be recovered from the immiscible fluid (e.g., oil) after polymerization. For example, the MOSs may be recovered by demulsficiation and/or de-emulsification, for example, by forming emulsified droplets and recovering the MOSs after the droplets are formed to remove any oil (and other contaminants). This may allow the cells to grow within the polymerized droplet (the MOS) without being inhibited by the immiscible fluid.

Although the methods and apparatuses described herein illustrate methods of forming the plurality of droplets, and thus the plurality of MOSs, by streaming the unpolymerized mixture into one or more streams of immiscible fluid (such as an oil or other hydrophobic material), the droplets may be formed by other methods that may allow for the size of the droplet to be controlled as described herein. For example, the droplets may be formed by printing (e.g., by printing droplets onto a surface). This may reduce or eliminate the need for an additional recovery step of emulsification/de-emulsification. For example, the droplets may be printed onto a surface, such as a flat or shaped surface, and polymerized. The droplets may be dispensed using pressure, sound, charge, etc. The droplets may be formed using an automatic dispenser (e.g., pipetting device) adapted to release the small amount of the unpolymerized mixture onto a surface, into the air, and/or into a liquid medium (including an immiscible fluid).

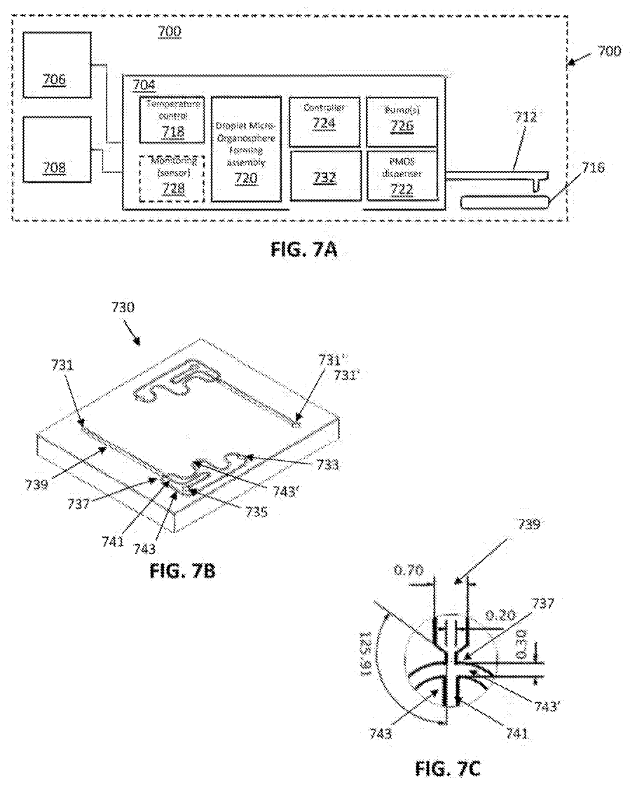

The method for forming the MOSs may be automated, or performed using one or more apparatuses. In particular, the method of forming the MOSs may be performed by an apparatus that allows the selection and/or control of the size of the MOSs (and therefore the density of the number of cells). For example, FIG. 7A illustrates one example of an apparatus 700 for forming MOSs as described.

In FIG. 7A, the apparatus typically includes an input for inputting either the unpolymerized mixture of the dissociated tissue sample and a fluid matrix material (already combined) or may separately receive the dissociated tissue sample, e.g., in a holding solution, and a fluid matrix material. The apparatus may include a holding chamber 706 for holding the unpolymerized mixture, and/or holding chambers (not shown) for holding the dissociated tissue (e.g., biopsy) sample and holding the fluid matrix material. Any or all of these holding chambers may be pressurized to control and/or speed up fluid flow out of the chambers and into the device. The apparatus may either receive the unpolymerized mixture or it may receive the components and mix it. The apparatus may control the concentration of the cells in the unpolymerized mixture and may dilute the mixture (e.g., by adding additional fluid matrix material to achieve a desired density. For example, the apparatus may include a sensor (e.g., an optical reader) for reading the density (e.g., the optical density) of the cells in the unpolymerized mixture (not shown). The sensor may also be coupled to the controller 724, which may automatically or semi-automatically (e.g., by indicating to a user) control the dilution of the cells in the unpolymerized mixture. The apparatuses may also include a port for receiving the unpolymerized mixture. The port may include a valve or may be coupled to a valve and the valve may be controlled by the controller 724 (or a separate controller).

The apparatus 700 may include a chamber 708 and/or port for holding and/or receiving the immiscible fluid. The immiscible fluid may be held in a pressurized chamber so that the flow rate may be controlled. Any of the pressurized chambers may be controlled by the controller 724 which may use one or more pumps 726 to control the pressure and therefore the flow through the apparatus. One or more pressure and/or flow sensors may be included in the system to monitor the flow through the device.

In FIG. 7A, the entire apparatus 700 may be enclosed in a housing 702 or a portion of the apparatus 704 may be enclosed in a housing. The housing may include one or more openings or access portions on the device, e.g., for adding the immiscible fluid and/or the unpolymerized mixture.

As mentioned, any of these apparatuses 700 may also include one or more sensors 728 for monitoring all or key portions of the manufacturing process. The sensors may include optical sensors, mechanical sensors, voltage and/or resistance (or capacitance, or inductance) sensors, force sensors, etc. These sensors may be used to monitor the ongoing operation of the assembly, including the formation of the MOSs. The apparatus 700 may also include one or more thermal/temperature regulators 718 for controlling the temperatures of either or both the immiscible fluid and/or the unpolymerized mixture (and/or the fluid matrix material).

Any of these apparatuses may also include one or more droplet forming assemblies 720 that may be monitored (e.g., using one or more sensors) as will be illustrated below in FIGS. 7C and 9. The droplet MOS forming assembly may include (or may be coupled with) a dispenser (e.g., a MOS dispenser) 722. The dispenser may dispense, for example, into a multi-well plate 716.

In general, the droplet MOS forming assembly 720 may include one or more microfluidic chips 730 or structures that form and control the streams of the unpolymerized mixture and forms the actual droplets. FIG. 7B illustrates one example of a microfluidic chip for forming MOSs 730. In FIG. 7B, the chip 730 includes a pair of parallel structures for forming MOSs. FIG. 7C illustrates the droplet-forming region of the microfluidic chip for forming MOSs, including an unpolymerized channel outlet 741 that opens (in this example, as a right angle) a “x” junction or region of intersection 737 to the channel outlet 741 and the immiscible fluid outlet(s), 743, 743′. The input from the immiscible fluid channel(s) may be at an angle relative to the angle (and point of intersection) with the unpolymerized material. In FIG. 7C, as in all figures in this description showing dimensions, the dimensions shown are exemplary only, and are not intended to be limiting, unless they otherwise specify.

In FIG. 7A, the microfluidics chip 730 includes an inlet (input port) 733 for the immiscible fluid into the chip (e.g., from the inlet port or storage chamber shown in FIG. 7A). A second inlet port 735 into the chip may be configured to receive the unpolymerized material and transport it down a semi-tortious path to the junction region. Similarly, the inlet port for the immiscible fluid, may be securely coupled to the outlet from the immiscible fluid chamber or inlet, described above.

The inlet port 735 for the unpolymerized material into the chip may be coupled through a delivery pathway 741 connecting the inlet to the junction region (as shown in FIG. 7C). Similarly, the inlet 733 for the immiscible fluid may connect to two (or more) connecting paths 743, 743′ to the junction region 737. A channel leaving the junction region 737 may pass the formed MOSs (in the immiscible fluid) down the channel to an outlet 731 that may connect to a dispenser (not show) for dispensing from the MOSs into one or more chambers, e.g., for culture and/or assaying.

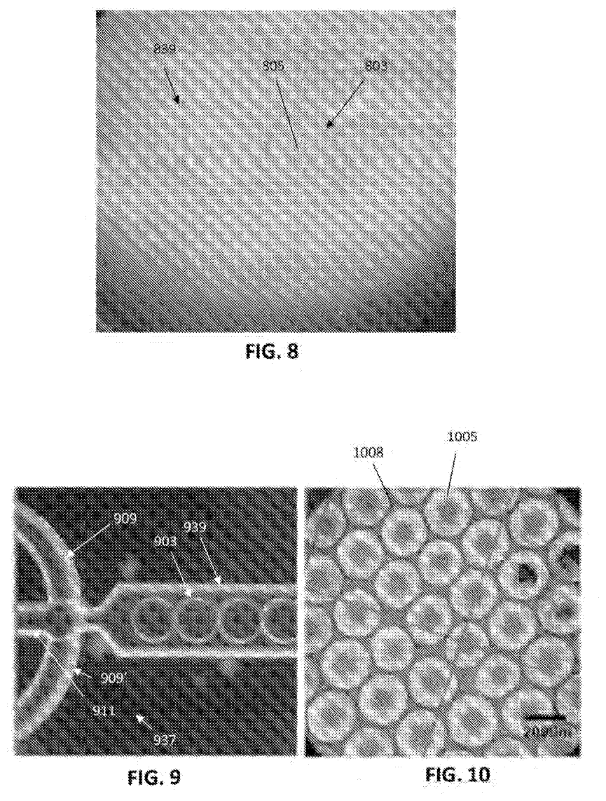

In the example shown in FIGS. 7B and 7C the formed droplets, which may become MOSs once polymerized, may be transmitted down a long, temperature controlled microfluidics environment, prior to being dispensed from the apparatus (not shown). For example FIG. 8 illustrates one example of a channel region 839 (e.g., element 739 in FIG. 7B) that is shown transparent, containing a plurality of MOSs 803 each containing a predetermined number of cell 805.

In FIG. 9, the junction region 937 is shaped as described above, so that the channel carrying the unpolymerized mixture 911 intersects one or more (e.g., two) channels 909 carrying a fluid, such as an oil, that is immiscible with the unpolymerized mixture. As the unpolymerized mixture is pressurized to flow at first rate out of the first channel 911, the flowing immiscible fluid in the intersecting channels, 909, 909′, permit a predefined amount of the unpolymerized mixture to pass before pinching it off to form a droplet 903 that is passed into the outlet channel 939. Thus, a minced (e.g., dissociated) clinical (e.g., biopsy or resected) sample of tissue, such as <1 mm in diameter, may be is mixed with a temperature-sensitive gel (i.e. MATRIGEL, at 4 degrees C.) to form the unpolymerized mixture. This unpolymerized mixture may be placed into the microfluidic device that may generate droplets (e.g., water-in-oil droplets) that are uniform in volume and material composition. Simultaneously, the dissociated tumor cells may be partitioned into these droplets. The gel in the unpolymerized material may solidify upon heating (e.g., at 37 degrees C.), and the resulting MOSs may be formed. This method may be used to produce over 10,000 (e.g., over 20,000, over 30,000, over 40,000, over 50,000, over 60,000, over 70,000, over 80,000, over 90,000, over 100,000) uniform droplets (MOSs) from the tissue (e.g., biopsy material). These MOSs are compatible with traditional 3D cell culture techniques. FIG. 10 illustrates a plurality of MOSS 1005 formed as described above, suspended in the immiscible material 1008 (e.g., oil).

In the exemplary microfluidics chip illustrated above, the junction is shown as a T- or X-junction in which the flow focusing of the microfluidics forms the controllable size of the MOS. Rather than a microfluidics chip, the droplets may be formed by robotic micro-pipetting, e.g., into an immiscible fluid and/or onto a solid or gel substrate. Alternatively, the droplets of unpolymerized material may be formed in the requisite dimensions and reproducibility by micro-capillary generation. Other examples of techniques that may alternatively be used for forming the MOSs in the specified size range and reproducibility from the unpolymerized material may include colloid manipulation, e.g., via external forces such as acoustics, magnetics, inertial, electrowetting, or gravitational.

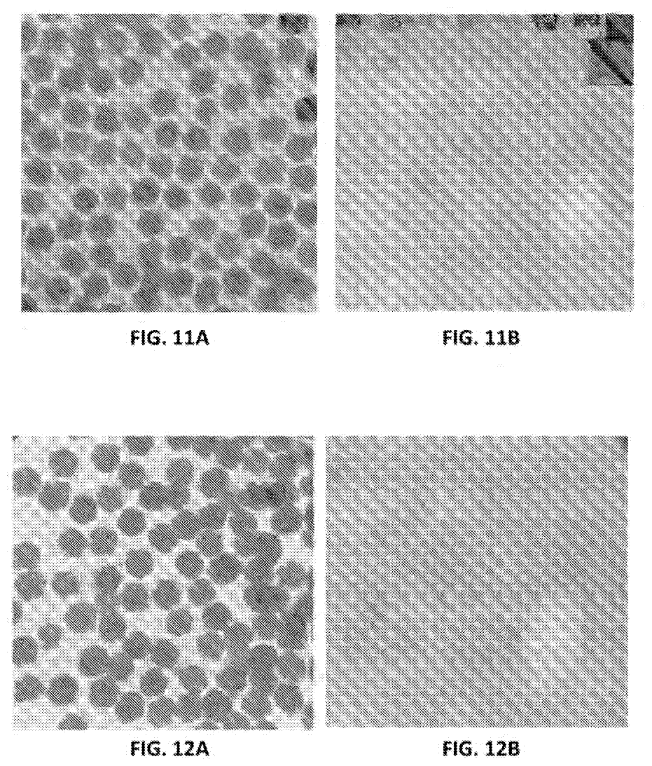

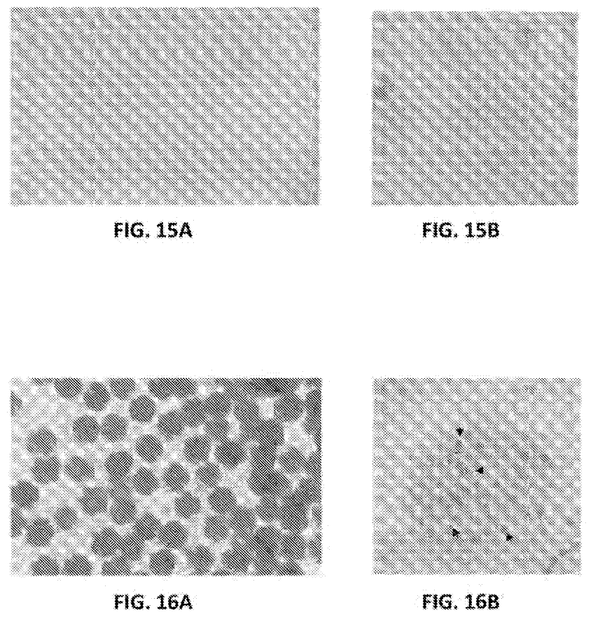

FIGS. 11A and 11B show examples of MOSs in oil formed as described above. The cells within these MOSs, derived from a single biopsy sample, are viable, as seen by vital dye staining, as shown in FIGS. 15A-15B and 16A-16B. For example, FIG. 12A-12B illustrates MOSs having tumor cells (similar to those shown in FIG. 11A-11B) that may be washed to remove the immiscible material (e.g., oil). This immiscible material may be removed relatively quickly after forming the MOS in order to prevent harm to the cells within the MOS.

As described in the examples below, MOSs described herein provide a good model for the effectiveness of various drug formations. A variety of IO drugs can be used and interact with MOSs. Example IO drugs include (but are not limited to) MAPK inhibitors (e.g., Vemnurafenib, Dabrafenib, PLX8349, Cobimetinib, Trametinib, Selurnetinib, and BVD-523), checkpoint inhibitors (e.g., T-cell targeted immunomodulators, Pembrolizumab, Avelumab, Durvalumab, Ipilimumab, TSR-022, MGB453, BMS-986016, and LAG525), other immunomodulators (e.g., anti-CD47 antibodies, and ADCC therapies), apoptosis inhibitors (e.g., ABT-737, WEHI-539, ABT-199) potential contributing pathways (e.g., Afuresetib, Idasanutlin, and Infliximab), chemotherapy agents (e.g., Cytarabine), cell therapy, cancer vaccine, oncolytic viruses, and bi-specific antibodies.

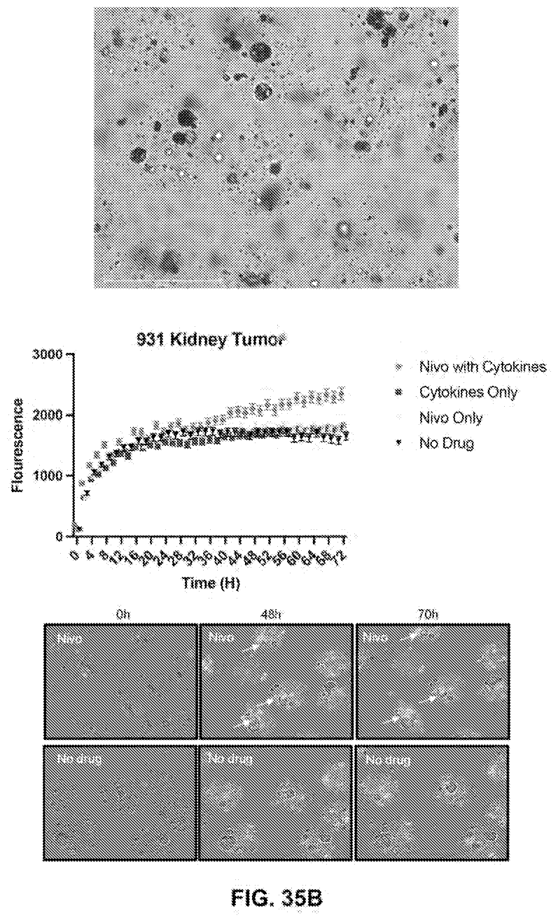

FIGS. 35A and 35B shows the efficacy of Nivolumab on MOSs derived from pulmonary and renal tumor biopsies. IO assays of the MOSs for these pulmonary and renal tumor biopsies were prepared with an Annexin V marker to indicate cellular apoptosis. As can be seen in FIGS. 35A and 35B, the MOSs display a good response to Nivolumab (in the form of tumor cell apoptosis). If this test were run on, for example, traditional bulk organoids, the results would not be meaningful (due to the lack of patient immune cells) leading to uncertainty in the best course of action for patient treatment,

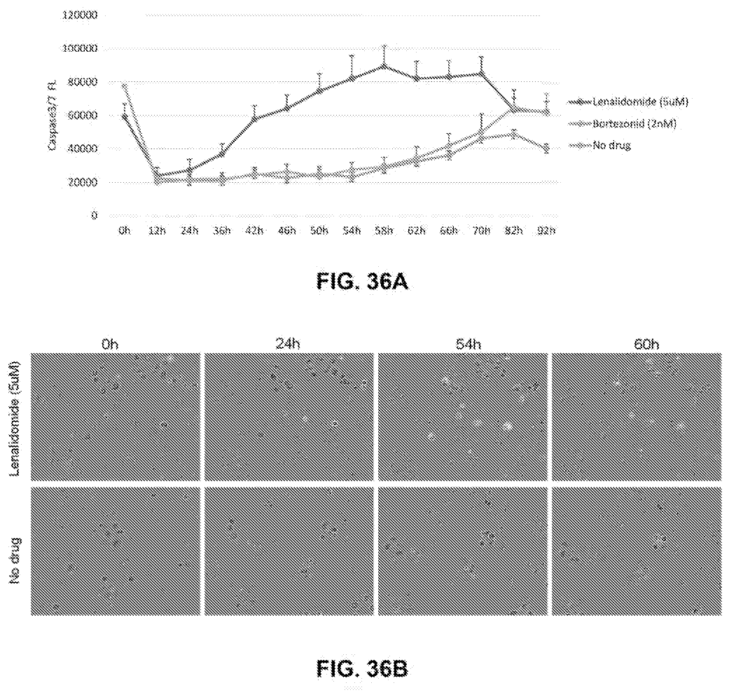

FIGS. 36A and 36B show the efficacy of Lenalidomide and Bortezoid on MOSs derived from multiple myeloma (MM) bone marrow biopsies. MM MOSs (on day 11) were treated with Lenalidomine (5 uM) or Bortezoid (2 nM). Caspase 3/7 green dye was added in the assay to monitor apoptosis. Incucyte images were taken every 2 hours for 4 days. As can be seen in FIGS. 36A and 36B, the MOSs display a good response to Lenalidomide (in the form of tumor cell apoptosis) but not to Bortezoid. This result would give a healthcare provider confidence that Lenalidomide is a better choice than Bortezoid for treating this particular MM patient. If this test were run on, for example, traditional bulk organoids, the results would not be reliable (due to the lack of patient immune cells) leading to uncertainty in the best course of action for patient treatment.

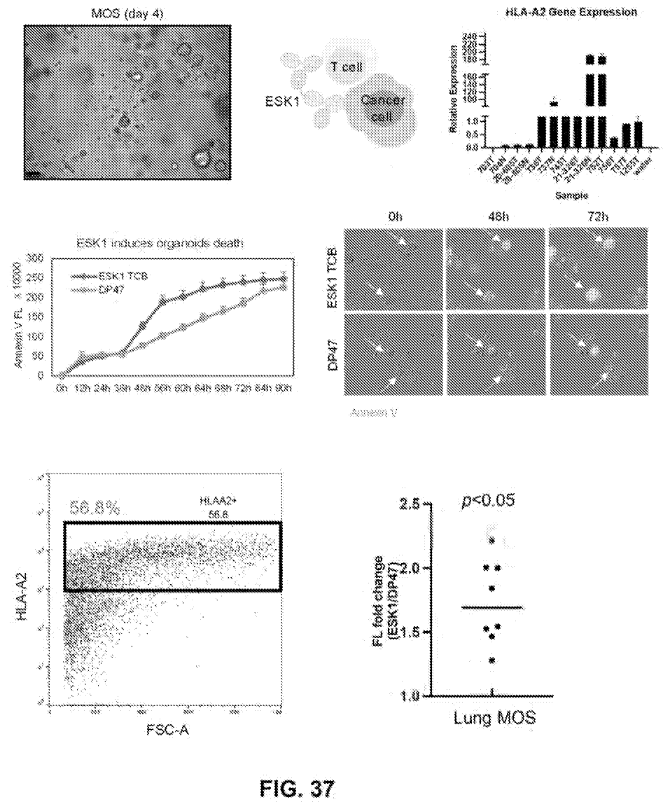

FIG. 37 shows the efficacy of ESK1 (a T-cell receptor-mimic antibody) on MOSs derived from a pulmonary Biopsy. IO assays of the MOSs for this pulmonary tumor biopsy were prepared with an Annexin V marker to indicate cellular apoptosis. As can be seen in FIG. 37, The MOSs display a good response to ESK1 (in the form of tumor cell apoptosis). If this test were run on, for example, traditional bulk organoids, the results would be less accurate (because ESK1 would not be able to reach its target) leading to uncertainty in the best course of action for patient treatment.

As described in the examples below, MOSs will readily uptake infused immune cells to provide a good model for the effectiveness of various immune cell therapies.

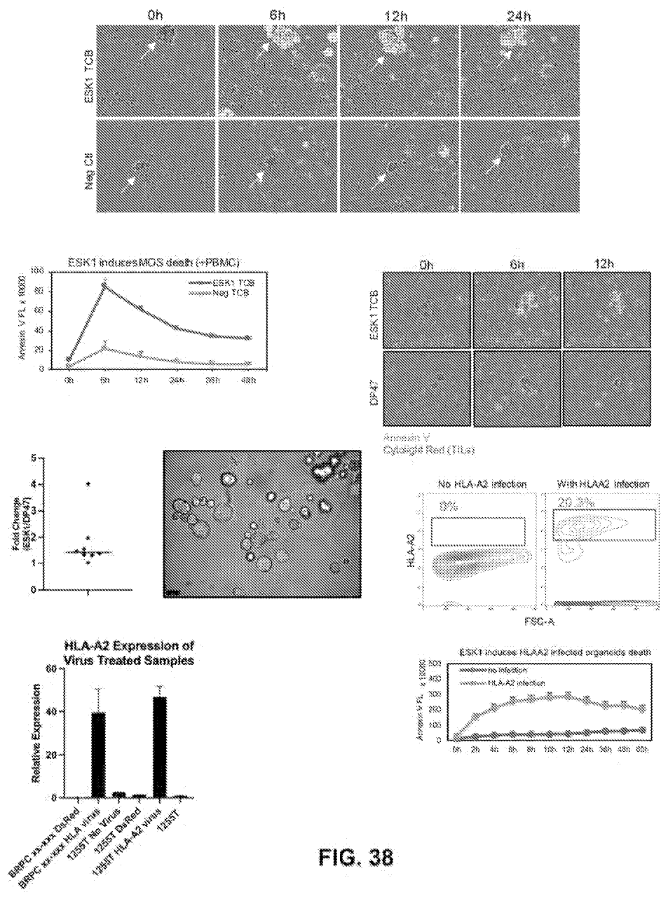

FIG. 38 shows the efficacy of ESK1 in combination with peripheral blood mononuclear cells (PBMC) on MOSs derived from a pulmonary tumor biopsy. IO assays of the MOSs for this pulmonary tumor biopsy were prepared with an Annexin V marker to indicate cellular apoptosis and Cytolight Red dye to indicate tumor infiltrating lymphocytes (TILs). As can be seen in FIG. 38, The MOSs display a good response to ESK1 with PBMC (in the form of tumor cell apoptosis). If this test were run on, for example, traditional bulk organoids, the results would be less accurate (due to the inability of PBMC to penetrate the traditional bulk organoids) leading to uncertainty in the best course of action for patient treatment.

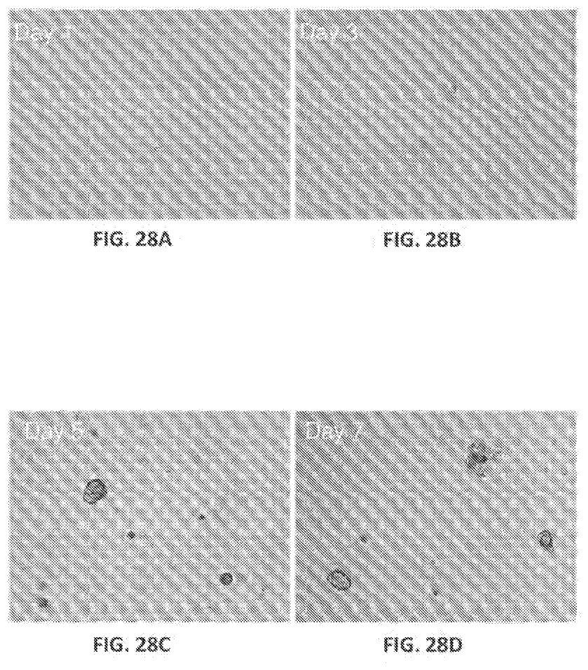

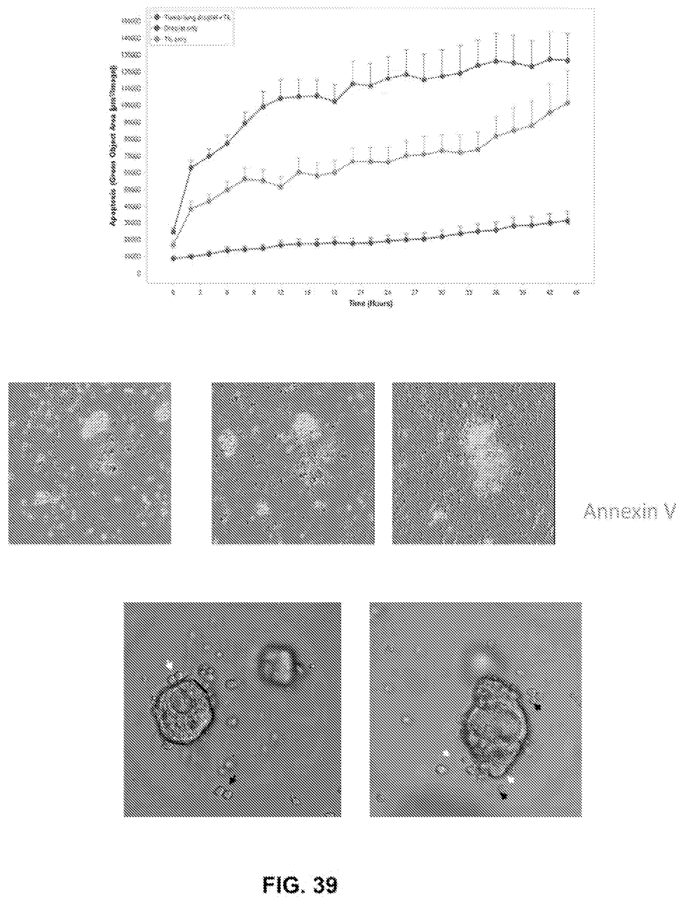

FIG. 39 shows the efficacy of TILs on MOSs derived from a pulmonary tumor biopsy, IO assays of the MOSs for this pulmonary tumor biopsy were prepared with an Annexin V marker to indicate cellular apoptosis. As can be seen in FIG. 28, The MOSs display a good response to TILs (in the form of tumor cell apoptosis). This is an indication that TILs can penetrate MOS to kill tumor cells.

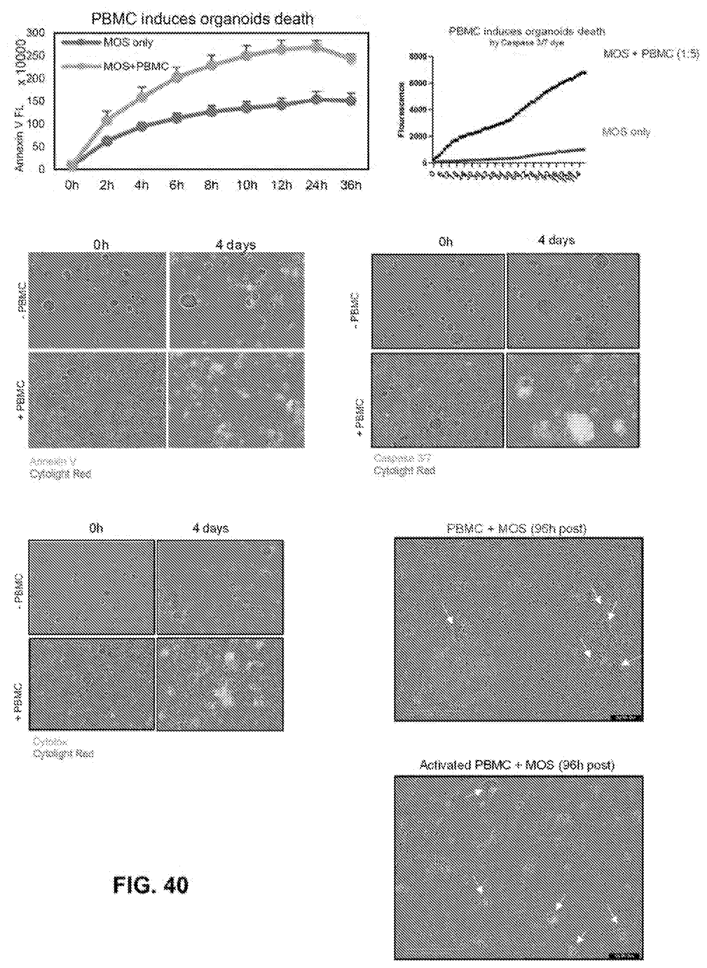

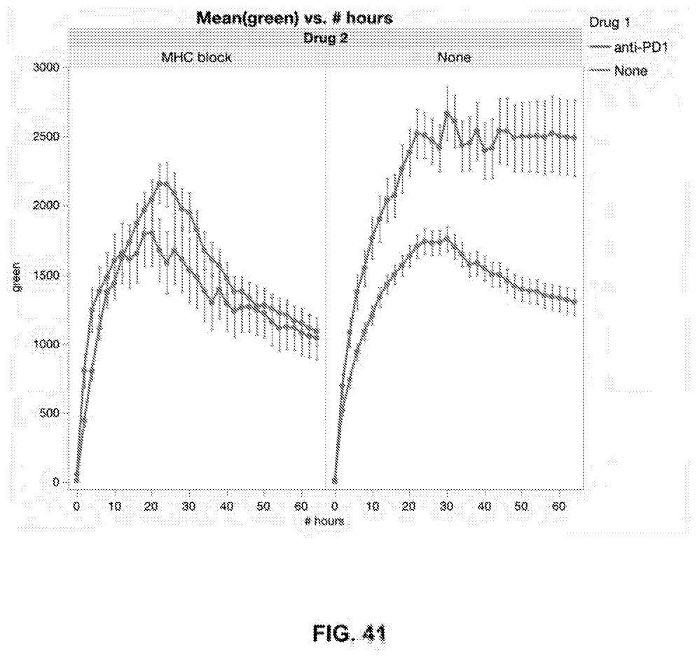

FIG. 40 shows the efficacy of PBMC on MOSs derived from a pulmonary tumor biopsy. IO assays of the MOSs for this pulmonary tumor biopsy were prepared with an Annexin V marker to indicate cellular apoptosis and Cytolight Red dye to indicate tumor infiltrating lymphocytes (TILs). As can be seen in FIG. 40, The MOSs display a good response to PBMC (in the form of tumor cell apoptosis).

FIG. 41 shows the efficacy of anti-PD1 (e.g., Nivolumab) in combination with TILs on MOSs. As can be seen in FIG. 41. The MOSs display a good response to anti-PD1 with TILs (in the form of tumor cell apoptosis). If this test were run on, for example, traditional bulk organoids, the results would be less accurate (due to the inability of TILs and nivolumab to penetrate the traditional bulk organoids) leading to uncertainty in the best course of action for patient treatment.

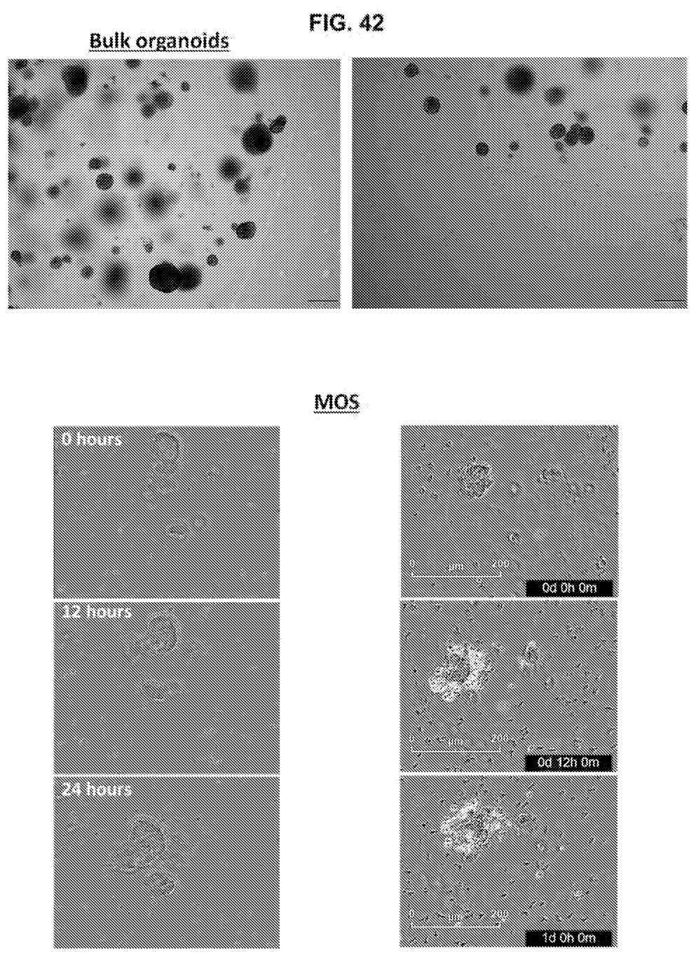

FIG. 42 shows the difference in the ability of infused patient derived T cells to penetrate bulk organoids vs. MOSs. As mentioned above, immune cells have difficulty penetrating traditional bulk organoids. This can be seen directly in FIG. 42 where T-cells are able to penetrate into the MOS and are unable to penetrate the Bulk Organoids.

The gel droplets may be recovered from the oil phase and resuspended, e.g., into PBS via PFO (perfluoro octanol) and centrifugation. This may separate the immiscible fluid from the MOSs. Thus, these MOSs, including tumor-based MOSs, can be successfully grown, as shown in FIGS. 1A-1C, 2A-2C, 3A-3C and 4A-4E, above, and in FIG. 13. This is an important improvement, as drug screening has to be performed on viable and growing primary tumor cells that retain their properties from patient tumors to predict patient outcomes. The high number and uniformity of these MOSs makes screening both possible and reliable, as will be described below.

In any of the microfluidic chips or devices described herein, the channels may be coated. For example, the channel of the microfluidic device may be coated with a hydrophobic material.

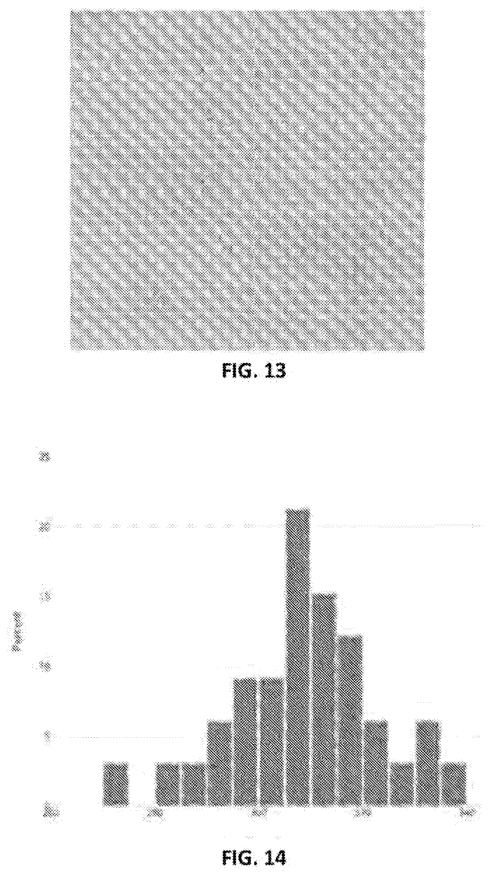

In general, the MOSs described herein are highly uniform in diameter, and may have a very low size, e.g., diameter, variance. This is illustrated, for example, in FIG. 14, showing a distribution of one example of droplet diameter sizes.

As mentioned, FIGS. 15A-15B shows MOSs formed as described herein; in FIG. 16A-16B, these MOSs have been stained with Trypan blue (arrowheads), showing that they are alive. The MOSs formed as droplets in this manner may contain growth-factors and matrix to mimic the biological environment from which the tissue arose. Patient samples (e.g., biopsy samples) may be formed into MOSs (including hundreds, thousands, or tens of thousands of MOSs) within a few hours of acquiring the tissue. The MOSs may have as few as 1 or between 4-6 cells (e.g., cancer cells when sampling a tumor) per MOS or as many as hundreds of cells. These methods have been shown to work for virtually all types of cancer and non-cancer tissues tested to date (n=32), including colon, esophagus, melanoma, uterus, sarcoma, renal, liver, ovary, lung, diaphragm, omentum, mediastinal lung, and breast cancer tissues. The MOSs may be cultured for any desired period of time, and typically show proliferation and growth in as few as 3-4 days. They may be maintained and passaged for months. As will be described in greater detail below, they may be used to screen thousands of drug compositions within as few as 4-6 days from taking the tissue (e.g., biopsy).

The MOSs described herein may, at any point after they are formed, be banked, e.g., by cryopreserving them. Tumor MOSs may be collected from many different patients and may be used individually or collectively to screen multiple drug formulations to determine toxicity and/or efficacy. Non-tumorous cells (healthy tissue) may be biopsied, banded and/or screened in parallel. Thus, these methods and apparatuses may allow for high throughput screening. The MOSs may be formed and allowed to passage twice (e.g., two doublings), and cryopreserved. As mentioned normal, healthy tissue may be used to form these same MOSs to generate hundreds, thousands, or tens of thousands of MOSs that may be used for assaying drug effects, drug response, biomarkers, proteomic signals, genomic signals, etc.

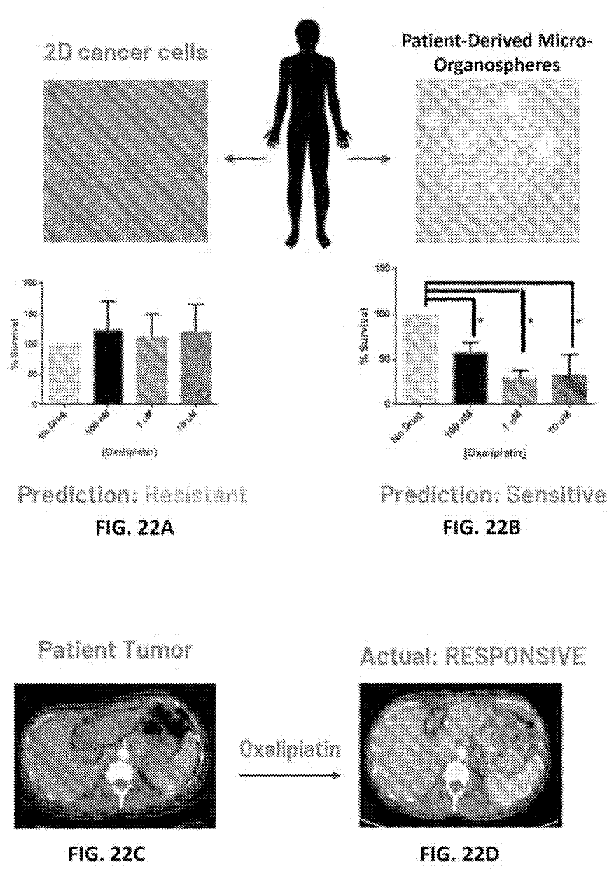

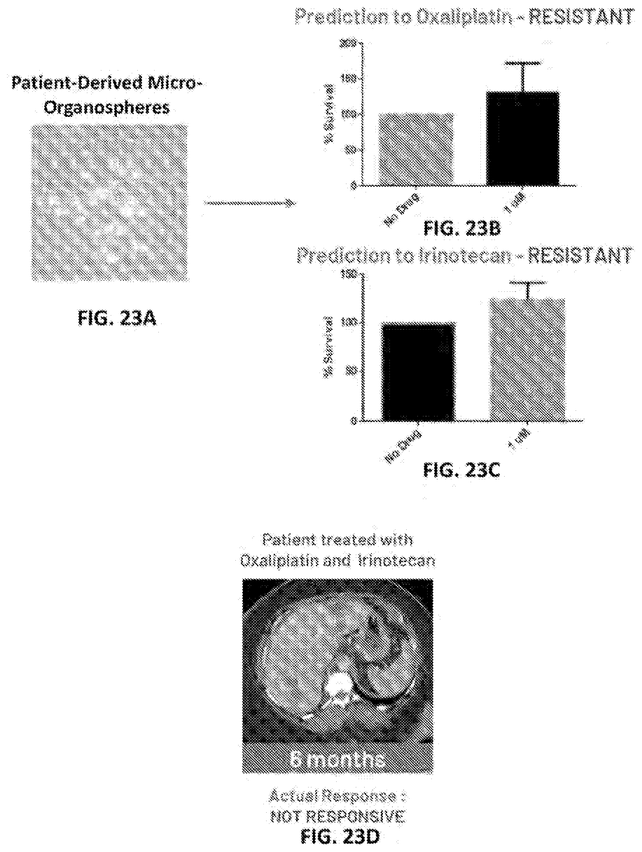

It is of particular significance that these MOSs survive in a biologically significant manner, allowing them to provide clinically and physiologically relevant data, particularly with respect to drug response, as will be described in FIGS. 22A-22D and 23A-23D. In particular, the MOSs described herein permit tissue extract/biopsy originated cells to grow exceptionally well and provide more representative data, especially as compared to organoids or spheroids. Without being bound by a particular theory, this may be because the cells may have a more constrained cell density in the MOSs, permitting cells to communicate without inhibiting each other while sharing signals. The MOSs also have a very large surface to volume ratio, more readily permitting transmission of growth factors and other signals to penetrate into the MOSs (e.g., the MOSs are less diffusion limited).

### Assays

The MOSs described herein may be used in a variety of different assays, and in particular may be used to determine drug formulation effects, including toxicity, on normal and/or abnormal (e.g., cancerous) tissue. For example, drug screening may include applying MOSs into all or some wells of multi-well (e.g., a 96-well) plate. Alternatively custom plates may be used (e.g., a 10,000 micro-well array may be formed of a 100×100 wells). The MOSs (e.g., gel droplets) may be applied into, or onto the multiple microwell arrays and incubated with culture medium. The MOSS may be cultured over the course of 3-5 days. In some circumstances, on day 5, the wells (e.g., micro-reactors) may then be dosed with drug compounds, e.g., based on a set of FDA-approved anticancer drugs, to examine the effects of the drug panel. For example, the drugs texted may be based on the National Cancer Institute (Division of Cancer Treatment and Diagnosis) screen, consisting of 147 agents intended to enable cancer research, drug discovery and combination drug studies. On Day 7, the MOSs may be imaged via standard fluorescent microscopy and ranked based on drug response.

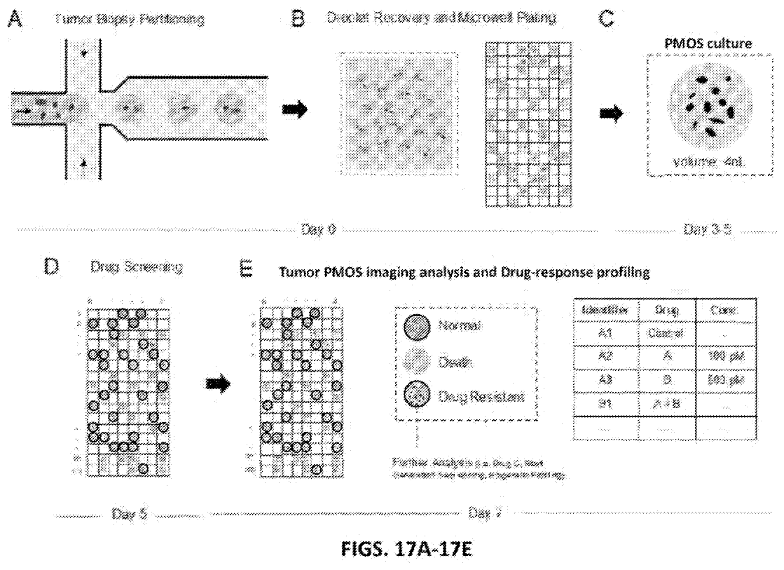

An example of this assaying technique is shown in FIGS. 17A-17E.

In this example, the screening assay may be automated. This may enable repeatable and automated workflow, which may increase the number of drugs screened from a few to hundreds. FIGS. 17A-17E illustrate one example of this workflow. In FIG. 17A, a tumor biopsy is taken and a plurality (e.g., >10,000) MOSs are formed as described above (in FIG. 17A the junction region forming the MOSs is illustrated). Thereafter, the MOSs may be recovered and washed (e.g., to remove the immiscible (e.g., oil) material in which they were formed). The MOSs may then be plated into one or more microwell plates. As shown in FIG. 17C, the MOSs may be cultured for one or more generations (e.g., one or more passages). This is shown occurring from day 0 to days 3, 4 or 5. Thereafter, the MOSs may be screened, as shown in FIG. 17D, e.g., by applying drugs to a subset of the replicant wells. Thereafter, as shown in FIG. 17E, on day 7, the cells in the MOSs may be imaged and/or automatically or manually scored to identify drug effects (e.g., drug screening and growth profiling).

The workflow shown in FIGS. 17A-17E may enable an integrated device to be used for growing, dosing and/or reviewing the MOSs. In one exemplary device, freshly biopsied or resected patient tumor samples may be disassociated and seeded into gel with regents to form the MOSs (as described above). A portion of the MOSs formed may be cryopreserved. The rest may be recovered and incubated until seeded into microwell plates for drug testing or screening as just described. Growth and viability assays may be performed on the MOSs, which may be imaged and tracked. Their response to drug treatments, such as IC-50, cytotoxicity, and growth curves, may be measured to identify effective therapeutics against the patient's tumor.

The methods and apparatuses described herein have numerous advantages, including reproducibility. The sample preparation process may be automated by the microfluidic sample partitioning which may reduce the need for specialized personnel for diagnostic testing and manual pipetting. This may be particularly helpful in a clinical setting. Moreover, this may enable uniformity among signal droplets, increasing assay sensitivity. In addition, these assays may minimize the time required to generate MOSs. Based on preliminary data, these methods may be able to generate a library of over 100,000 MATRIGEL-tumor droplets (MOSs) in less than about 15 minutes. These methods are also highly scalable, and can be multiplexed to run multiple patient biopsies in parallel.

Finally, these methods are flexible and compatible with other techniques. As a research tool, droplet-based microfluidics is generally compatible with a wide range of hydrogel materials such as agarose, alginate, PEG, and hyaluronic acid. As such, the starting gel composition can easily be modified to accompany and encourage MOS growth. Moreover, the droplet-size can be adjusted by modifying the size of our microfluidic device. Together, these allow a large selection of gel material composition and micro-reactor sizes.

The miniaturized assays described here, e.g., using the MOSs, may maximize the patient tumor biopsy, enabling more drug compounds to be screened. For example, a 600 uL tumor sample can be partitioned into ˜143,000 individual micro-reactors that are ˜4 nL in volume. By maximizing the tissue sample, multiple experimental replicates may be examined, increasing statistical power. These techniques may allow the inspection of intra-tumor heterogeneity, drug perturbation and identify rare cellular events, such as drug resistance. The MOSs may generally be compatible with downstream assays including single cell RNA transcriptome analysis and epigenetic profiling. In addition, by maximizing the tissue (e.g., biopsy) sample efficiency as provided by the MOSs, a portion of the MOSs may be banked (e.g., by cryopreservation for biobanking) for future novel drug assays and/or for confirmation analysis, including genetic screening.

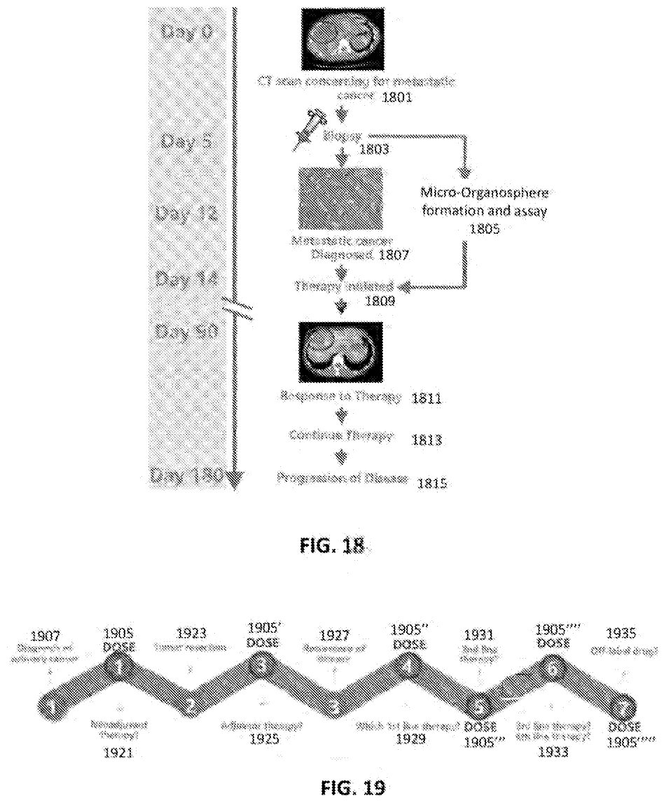

For example, FIGS. 18 and 19 illustrate therapeutic methods that use the methods and apparatuses, including the MOSs, described herein. For precision and personalized medicine, these methods and apparatuses can be used as a clinical indicator for appropriate drug selection to improve clinical outcome and drug response. As one example, a patient diagnosed with metastatic cancer will take a biopsy for histopathology and for screening of a plurality of MOSs formed from a biopsy as described herein. Within 7˜10 days, the screening may be performed from the biopsy to identify the most effective standard-of-care therapy so the patient can start treatment around 14 days.

An example of this is illustrated in FIG. 18. In this example, the tumor may be identified at day 0 (e.g., by CT scan) 1801, and a biopsy taken 1805 at day 5, and on the same day hundreds, thousands, or tens of thousands of MOSs can be formed and cultured for 1-5 days and screened 1805 to identify one or more drug compositions that can be used. This same step (forming the MOSs and screening) may be used to guide precision medicine at multiple clinical decision points throughout disease progression. In this example, therapy using the identified one or more drug compositions may be started on day 14 (1809), and the patient may later be monitored during the course of treatment (e.g., a follow-up CT scan on about day 90) to confirm that the tumor is responding to the treatment 1811. If so, the therapy may be continued 1813 and the ongoing progress monitored 1815.

As mentioned, the use of MOSs to assay may be repeated at multiple point throughout treatment and during the course of the treatment. This is illustrated in FIG. 19. For example, when a patient is first diagnosed 1907 with a resectable primary tumor, this technique (e.g., generation of MOSs and screening 1905) can be used to determine the most effective neoadjuvant therapy 1921. Thus, a biopsy may be taken and hundreds, thousands, or tens of thousands of MOSs may be formed and screened with a panel of potential drug compositions. Once the primary tumor is resected 1923, this technique 1905′ may indicate whether and which adjuvant therapy should be chosen 1925. If recurrence or metastasis happens after the surgical removal of the primary tumor 1927, the same technique (e.g., generating and screening MOSs from a fresh biopsy 1905″, 1905′″, 195″″) can be used to guide standard-of-care therapy, including 1st 1929, 2nd 1931, and 3rd line 1933 therapies. If the patient eventually becomes tolerant or resistant to all standard-of-care therapy, this technique 1905″″″ can be performed to identify off-label drugs to treat resistant tumors 1935. This technique can also be used as companion diagnostics to identify patients for a specific treatment. Lastly, the technique can be used to derive and preserve patient-derived MOSs to establish an Organosphere-based living cancer bank for screening, genomic profiling, new drug discovery, drug testing and clinical trial design.

Because these techniques, and the generation of a huge number of MOSs may be done with relatively low-invasiveness (e.g., by resection or biopsy), to provide reasonably fast results from the screening, these methods may be easily adapted for standard of care. For example, the volume of cellular material from the assue (e.g., biopsy) input is quite small, and may be dissociated into a volume of, e.g., between 10 μL to 5 ml.

In general, the use of the MOSs described herein for screening may be automated or manually performed. Virtually any screening technique may be used, including imaging by one or more of: confocal microscopy, fluorescent microscopy, liquid lens, holography, sonar, bright and dark field imaging, laser, planar laser sheet, including high-throughput embodiments of image-based analysis methods (e.g., using computer vision, and/or supervised or unsupervised model, e.g., CNN). Downstream screening may include sampling the culture media and/or performing genetic or protein screening (e.g., scRNA-seq, ATAC-seq, proteomics, etc.) on cells from the MOSs.

### EXAMPLES

### Example 1

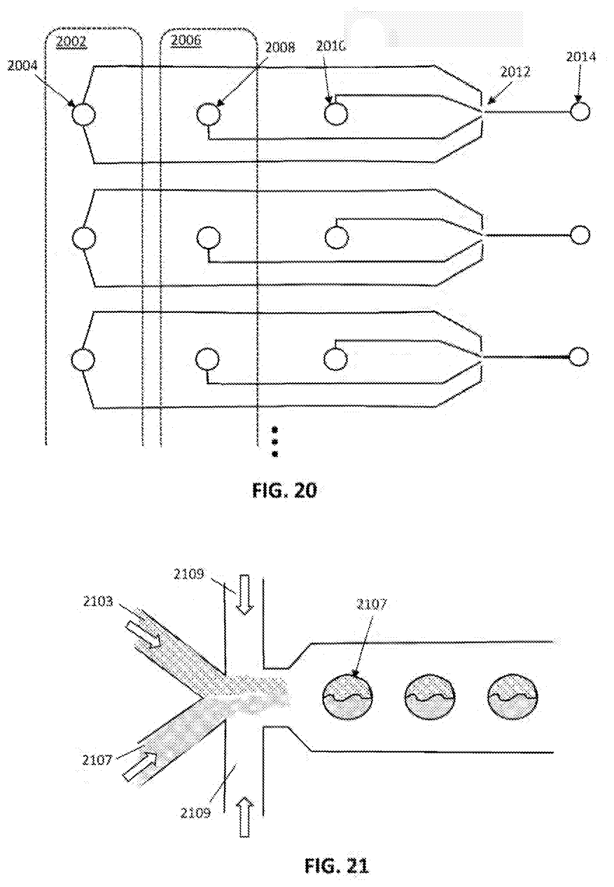

FIGS. 20 and 21 illustrate another example of an apparatus for forming a plurality of MOSs as described herein. In FIG. 20, the apparatus may include a plurality of MOS forming junctions, in which the immiscible material (e.g., oil) 2002 may be added to a reservoir and/or port 2004 in the device. Similarly, the unpolymerized material 2006 (in this example, including the dissociated biopsy cells and the fluid matrix material) may be added to the reservoir or port 2008 in the apparatus. A second or additional material (e.g., a biologically active agent) may be added via a third set of ports 2010. These components may be combined at a junction (similar to that described above) forming a droplet in the immiscible material that may be polymerized into the MOSs. In FIG. 20, three (or more) parallel junctions with corresponding inputs and output are shown.

FIG. 21 illustrates the method of forming the MOSs using an apparatus as shown in FIG. 20. In this example the resulting MOSs include both the target (e.g., tumor) biopsy cells and also one or more additional biologically active agents that are combined to form the MOS. For example, a first channel 2103 may include the unpolymerized material (including the dissociated biopsy cells and the matrix material), a second channel 2107 includes an additional active biological material, and a pair of intersecting channels 2109, 2109′ carrying the immiscible material (e.g., oil) converge at the junction to form the size-controlled droplets that are polymerized to form the MOSs 2107.

In this example the additional active biological material may be, e.g., freezing medium (e.g., to aid in banking the MOSs), and/or co-cultures with additional cells (e.g., immune cells, stromal cells, endothelial cells, etc.), additional supportive network molecules (e.g., ECM, collagen, enzymes, glycoproteins, biomimetic scaffolds, etc.), additional growth factors, and/or drug compounds.

### Example 2: Screening Results

As mentioned above, the MOSs and methods of using them to screen for drug compositions may be used to accurately predict the response of a patient tumor to one or more drug therapies. In some cases, the use of MOSs may provide accurate results where traditional cultured drug screening does not accurately predict drug response. For example, in FIG. 22A-22D, the MOSs, but not a cell line, was able to correlate with patient response. In FIG. 22A, a traditional cell line dosed with drugs (e.g., Oxalipalitin) was examined; the cell line showed no effect, predicting that the tumor would be resistant to the drug at all dose ranges examined.

For comparison a plurality of MOSs were generated from a patient biopsy, as shown in FIG. 22B. In this example, the MOSs showed significant decreases in cell survival from the tumor MOSs, predicting drug sensitivity. In fact, when treated with the drug, the tumor responded to treatment, as shown in FIGS. 22C (before treatment) and 22D (post treatment).

### Example 3: Correlation Between MOSs and Patient Response

In a similar set of experiments, MOSs were generated from biopsy material (FIG. 23A), and a drug effect screen was performed using the resulting MOSs. FIG. 23B shows the effect of a first drug (Oxalipalatin) on these MOSs, showing no change in the percent survival of the MOSs in the presence of the drug, predicting drug resistance. Similarly, treatment with a second drug, Irinotecan, showed a lack of effect on the MOSs, predicting drug resistance, as shown in FIG. 23C. The patient was treatment with both Oxalipalatin and Irinotecan, and, after 6 months of treatment, showed no response. Thus, the MOSs correlated strongly with patient response to the standard of care drugs. In this case, the patient endured six months of side effects and toxicities that may have been avoided by the predicted response from the MOSs, indicating (within 7-10 days from the biopsy) that the tumor would not respond to these drugs.

### Example 4: Multi-Drug Screening

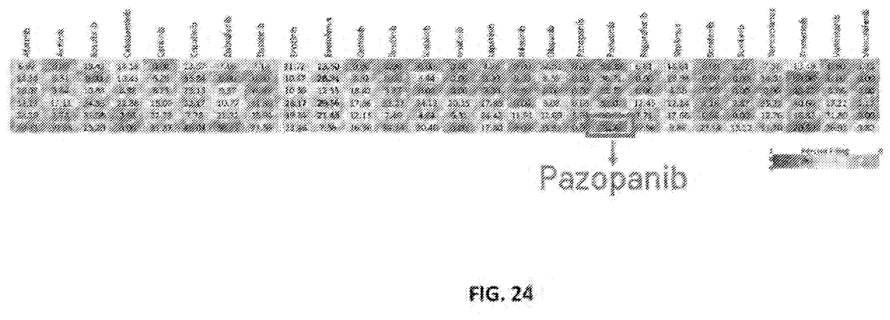

FIG. 24 shows an example of a panel of drug (e.g., chemotherapeutic agents) that may be generated using a Patient Derived plurality of MOSs as described herein. In this example, a drug screen using the Patient-Derived MOSs was run by dosing a plurality of replicates for each of a plurality of (27) drugs. A single tumor biopsy was used to generate a plurality of MOSs in large quantities extremely fast (e.g., within less than two weeks) and these MOSs were tested against the panel of drug formulations (e.g., 27 formulations are shown). This testing was done in parallel and could be automatically quantified (e.g., by optical detection and quantification). In this example, the drug showing the largest toxicity was Pazopanib for this particular tumor.

Combinations of drugs as well as different drug concentrations may be examined in parallel. As hundreds, thousands, or tens of thousands of MOSs may be generated from the same tumor biopsy, array testing of this sort is made practical by the methods and apparatuses described herein.

### Example 5: Biopsy Sample Preparation

Materials: an apparatus for forming the MOSs, as described above, including a droplet microfluidic chip (200 um); Bio-rad Droplet Generation Oil for EvaGreen (catalog #186-4006), 3-5 mL per run, Perfluoro octanol (PFO), Sigma, 10% Perfluoro octanol (PFO) in Novec HFE 7500, PBS, Cell culture media (i.e. RPMI w/10% FBS and 1% PenStrep), 70 um or 100 um filters, 50 mL conical, Petri dish.

Biopsy sample dissociation: using a biopsy sample (human/animal) to generate a dissociated sample (i.e. single cell tissue) from patient. Coat the microfluidic chip, and assemble the microfluidic chip and holder. Connect microfluidic tubing and fitting to an output (e.g., multiwall plate, 15 mL Eppendorf, etc.) for the MOSs and the waste oil.

Run the device to form the MOSs. Remove the output (e.g., plate, Eppendorf tube, etc.) containing the droplets from the incubator (after at least 15 minutes). Remove any excess oil from the output. The droplets should be buoyant, so the oil should be at the bottom of the vial. Be careful not to remove the droplets from the tube. Add 100 uL of 10% (v/v) PFO to the output. Carefully swirl and wait ˜1 min. Do not pipette or disturb the sample. Centrifuge at 300 g for 60 sec. Remove the supernatant (excess oil/PFO). Do not pipette or disturb the sample. Remove as much of the PFO as possible, as this chemical can reduce cell viability during culture. Add 1 mL of cell culture media. Do not pipette or disturb the sample. Centrifuge at 300 g for 60 sec. Remove supernatant and any excess oil/PFO. Add 1 mL of cell culture media. Carefully pipette the sample up and down (˜30 times) with a 1 mL pipette tip. Be careful not to over pipette or disturb the droplet sample. Using a 1 mL pipette tip, place the droplet-media solution through the 70 um or 100 um filter (connected to a 50 mL conical). Some droplets will stick to the inside of the output (e.g., a 15 mL Eppendorf). Rinse each tube with 2-3 mL of PBS and pipette up and down. Place rinsed PBS and droplets through the filter. Repeat this step twice, or until the tube looks clear, and the droplets have been transferred to the filter. Using a 1 mL pipette tip, carefully wash the filter containing the droplets with ˜5 mL of PBS. Try and cover the entire surface area of the filter. This washing step removes any excess oil and PFO from the sample, and allows the final recovery of the gel droplets into cell culture media.

Once drained correctly (˜1-2 minutes), carefully remove the filter from the 50 mL conical. Flip the filter upside-down and wash the back side with fresh cell culture media, and catch the solution in a fresh petri dish. This detaches the droplets from the filter, and places them in the cell culture media. It is recommended to use a 1 ml pipette tip, and wash with ˜5 mL of media

Check the quality of the droplets under the microscope. Most/all of the oil should be removed. If poor recovery, the sample can be re-filtered. Density of MOSs recovered may be checked by hemocytometer

### Example 6: Renal Tissue MOSs

In another example, MOSs may be formed from biopsied renal tissue. For example, instruments used may include: a tube rotator or 100 μm and 70 μm cell strainer, 15 mL conical tubes, 50 mL conical tubes, Razor blades, Tweezers and surgical scissors, Petri dish (100×15 mm) or tissue culture dish. The reagents may include: EBM-2 media, Collagenase (5 mg/ml stock), Hank's Balanced Salt Solution (HBSS), Calcium Chloride (10 mM stock solution), Phosphate Buffer Solution (1×PBS), MATRIGEL, 0.4% Trypan Blue solution and Trypsin.

Rental tissue to be stored in a cold transport media and on ice at all times. 2 mL of enzymatic digestion solution may be placed in a 15 mL conical tube. Add 600 uL of calcium chloride (final concentration: 3 mM) and add 200 uL of collagenase (final: 0.5 mg/mL). Transfer the renal sample into a petri/culture dish. Remove all excess or non-tumor tissue with sterile tissue or razor blade. Add 1 mL of the enzymatic solution to the tissue. Mince the sample into small pieces with the sterile razor blade (<2 mm2). Hold down the plate with tweezers or by hand. Transfer minced tissue and enzymatic solution back into the 15 mL tube with the enzymatic solution. Place the tube in the tube rotator or a 15 mL tube rotator between 30-60 minutes in 37° C. incubator. Remove the tube from the incubator. Quench the enzymatic digestion with at least 6 mL EBM-2 (at least 3 times the amount of enzymatic digestion solution). Pipette to mix. Place a 100 μm or 70 μm cell strainer onto a 50 mL conical tube. Transfer sample through the strainer. Transfer solution to a new 15 mL conical tube. Centrifuge the sample at 1500 rpm for 5 minutes. Discard the supernatant, leaving the cell pellet. Resuspend the pellet in 1 mL EBM-2 media. Add 10 μL cell mixture to 10 μL of Trypan Blue on a piece of parafilm and transfer to a cell counting plate or hematocytometer. Calculate cell concentration (#/mL). Centrifuge at 1500 RPM for 5 minutes and discard the supernatant, leaving the pellet. Resuspend cell pellet in 50 uL of MATRIGEL per 1.25×105 cells. Perform on ice. Plate 50 uL domes of MATRIGEL-cell suspension in the center of wells in a pre-warmed 24-well flat bottom plate. Transfer the plate to a 37° C. cell incubator and incubate for at least 20 minutes. Confirm that domes are polymerized. Gently add 500 μL of prewarmed EBM-2 media down the wall of the well. Incubate in 37° C. incubator. Perform a full media change every 2 days to expand MOSs.

### Example 7: Liver MicroOrganoSphere

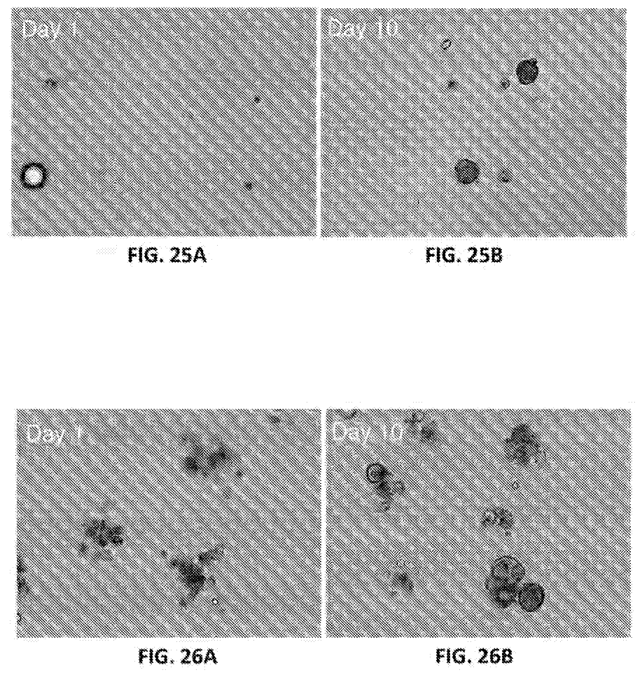

As mentioned, MOSs may be formed from normal (e.g., non-cancerous) and/or abnormal tissue. For example, FIGS. 25A-25 and 26A-26B illustrate one example of MOSs formed from a mouse liver tissue that has been dislocated, combined with a fluid matrix material to form an unpolymerized mixture, then a droplet of the unpolymerized mixture was polymerized to form the MOSs. In this example, the MOSs have a diameter of about 300 μm. In FIGS. 25A-25B the MOSs were formed with a single cell per droplet. In FIGS. 26A-26B the MOSs were formed with 25 cells per droplet. In FIG. 25A the MOSs are shown one day after forming; FIG. 25B shows the MOSs after ten days in culture. Cells in some of the MOSs have divided, forming clusters exhibiting structure; other MOSs included cells that were slower to divide or that did not divide. Similarly, in FIGS. 26A-26B the MOSs initially including about 25 cells in each MOSs. After ten days in culture, some of the MOSs showed a great deal of cell growth, forming structures, while other MOSs showed only modest growth. In both cases, the cells within the MOSs have been found to exhibit properties characteristic of the original tissue (e.g., liver cells) from which they originated.

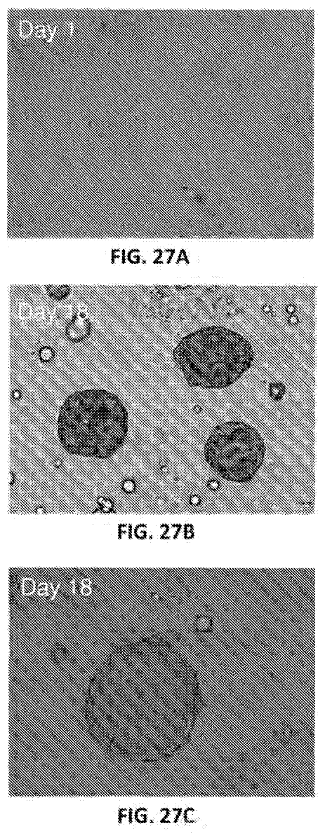

The same procedure was successfully performed on human liver tissue, as shown in FIGS. 27A-27C. In this example the MOSs were initially formed with about fifty cells, as shown in FIG. 27A. By day 18 in culture, some of the MOSs showed cells having clusters and forming structures, while others had smaller structures or the cells did not divide.

### Example 8: Cultured Cell MicroOrganoSpheres

In addition to primary tissues, e.g., removed from a patient immediately or shortly before forming MOSs, MOSs may be formed from cultured cells or cells, including either 2D cultured cells or 3D cultured cells.

The MOSs may be formed from cell lines grown as part of a Patient Derived Xenograft (PDX). For example, FIGS. 28A-28D illustrate MOSs formed from cultured PDX240 cells. PDX240 cells are a Patient Derived Xenograft (PDX) tumor cell line (numbered 240 based on patient source) that were human tumors grown in immunodeficient mice (PDX) to form in vivo tumors. The xenograft tissue was extracted dissociated, and used to form MOSs as described above. In this example a single cell was included in each MOS as it was formed. FIG. 28A shows the MOSS after one day in culture, while FIG. 28B shows the MOSs after three days in culture and FIGS. 28C and 28D show the MOSs after five and seven days in culture, respectively. With progressive time in culture, at least some of the MOSs show the cells dividing and forming structures.

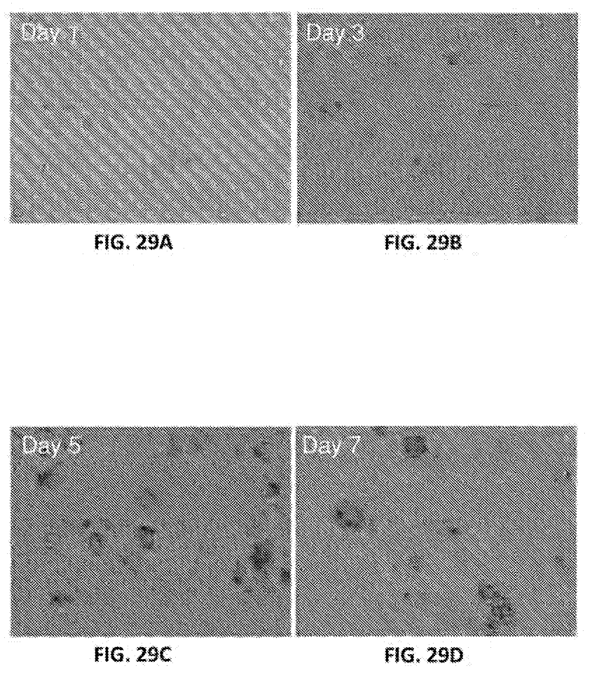

FIGS. 29A-29D show a similar experiment in which five PDX240 cells were initially included in each droplet forming each of the MOSs. With time in culture (e.g., from day 1, day 3, day 5 and day 7, as shown in FIGS. 29A-29, respectively) the cells may divide and form structures.

### Example 9: Comparison of MOSs with Traditional Organoids

Organoids were formed from Patient Derived Xenograft cells (including the PDX240 cells described above and a second PDX cell line, PDX19187) and were compared with MOSs formed using the same cells. The organoids were formed using conventional techniques in which a large mass of MATRIGEL in a well or dish was seeded with cells and cultured until growth was confirmed. MOSs were generated from the traditional organoids.

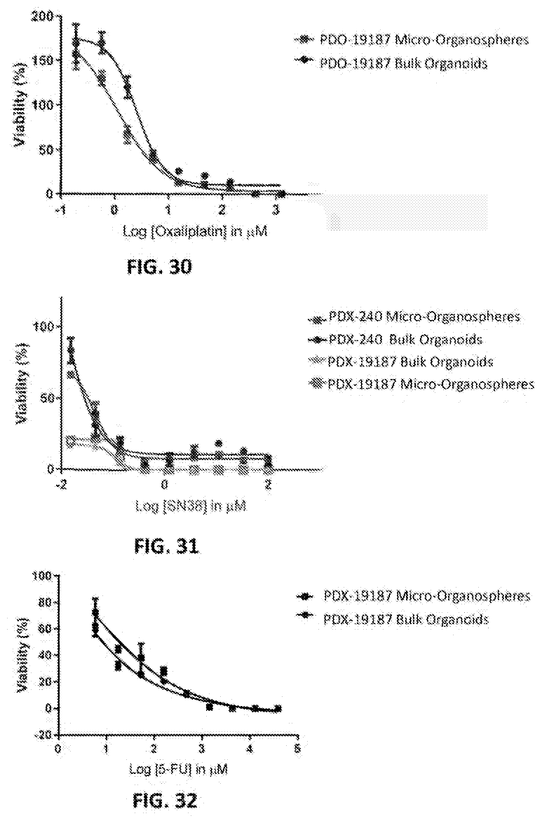

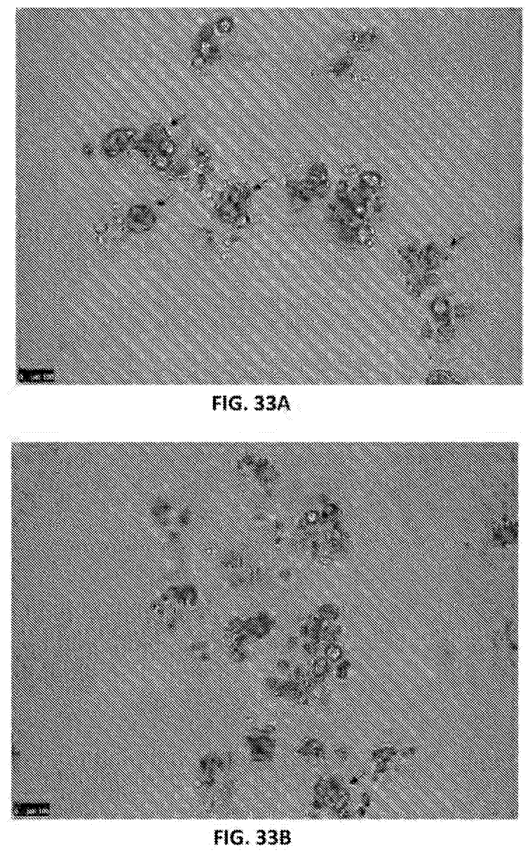

Both traditional (“bulk”) organoids and the MOSs were then treated with the same drugs (e.g., Oxaliplatin or SN38) and cell viabilities were measured after 3 days of treatment. The drug response curves shown in FIGS. 30 and 31 were generated, and show similar response curves. For example, in FIG. 30, the drug response curves of PDO19187 bulk organoids and MOSs showed similar response curves to Oxaliplatin concentration, as did PDX240 bulk organoids and MOSs. In FIG. 31, the drug response curves for both PDX19187 and PDX240 also showed similar results for both bulk organoids and MOSs for SN38. FIG. 32 shows response curves for another anti-cancer drug, 5-FU (Fluorouracil), again showing similar drug response curves for both PDZ-19187 and PDX-240 traditional organoids and MOSs.

Thus, the MOSs described herein, which may be formed more quickly and reliably, and which may have a higher overall survival rate as compared to traditional organoids, may provide drug responses that are comparable to those of bulk organoids formed using the same cells. However, as described herein, the MOSs may be used more quickly and may be formed in much larger numbers.

### Example 10: Drug Effects on MOSs

In general, the MOSs described herein may be used to perform one or more assays, including toxicity assays. Any appropriate assay may be performed, as the results determined by analysis of the tissue (e.g., cells, tissue structures) suspended within the MOSs. The MOSs described herein may be assayed or analyzed optically, chemically, electrically, genetically, or in any other manner known in the art.

Optical (either manual or automatic) detection may be particularly useful and may include optically analyzing the effects of one or more drug formulations on the tissue (including cells, clusters of cells, structures of cells, etc.) within the MOSs. As mentioned above, the drug formulation may be assayed for cell death (e.g., number and/or size of tissues) within the MOSs tested. The MOSs may be assayed for cell growth, including reduction in the size, type and/or rate of growth. The MOSs may be assayed for changes in the tissue structures formed.

For example, FIG. 33A-33B illustrate the effect of one drug formulation, in this example, acetaminophen (10 mM) on mouse liver MOSs. FIG. 33A is a control group, in which the MOSs were not treated, showing tissue within the MOSs (arrows) grown when cultured. FIG. 33B shows a similar set of MOSs formed from mouse liver that were instead treated with 10 mM acetaminophen. In the control group the tissue structures within the MOSs are relatively large as compared with the treatment group. The tissue in most of the MOSs of the acetaminophen group is smaller and contains many dead cells.

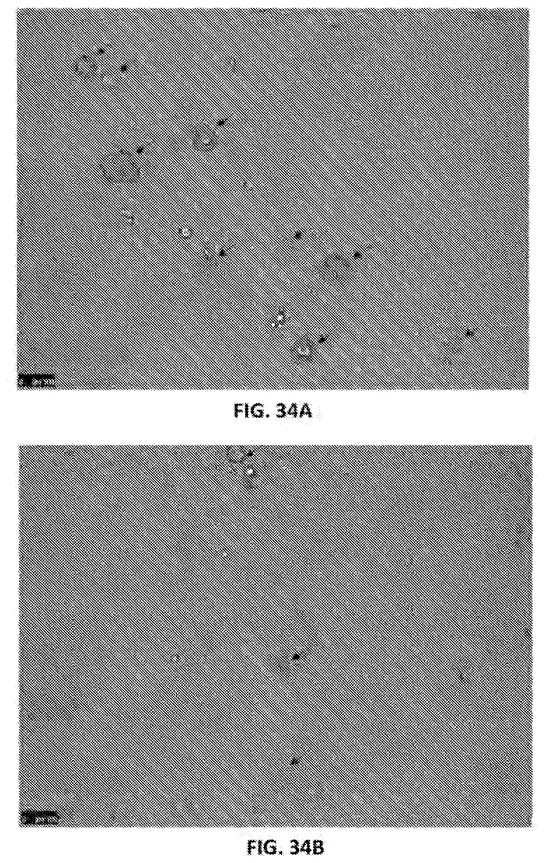

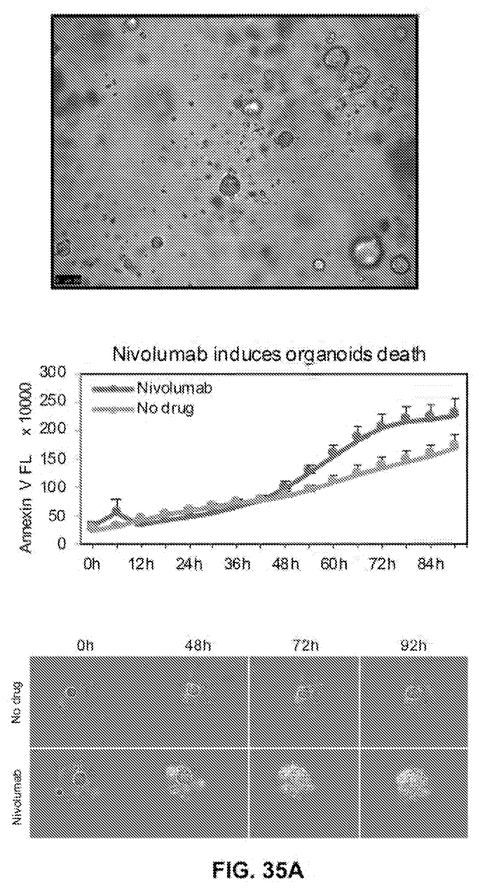

Similarly, FIGS. 34A-34B also show toxicity assays using human liver MOSs. FIG. 34A shows typical human liver MOSs observed in the control group including tissue structures (indicated by the arrows) formed therein. FIG. 34B shows the treatment group, in which the human liver MOSs are treated with acetaminophen (10 mM). The tissue in the treated MOSs showed a significant increase atypical tissue structures (arrows) and debris, as compared to the control group.

Any of these reviews, including optical reviews, may be scored, graded, ranked, or otherwise quantified. For example, in FIGS. 33A-33B and 34A-34B, the results of these two assays may be quantified to indicate the size difference, number of live/dead cells/tissue, and the like. The scoring may be automated.

### Example 11: Viral Vector-Based Delivery of Components into a MOS

In general, any viral vector-based method, including those described herein, may be utilized to deliver components into a MOS.

In this example, the below protocol was followed:
MOS were generated with colorectal cancer (CRC) patient derived organoids cells in 70% matrigel, 10 cells per MOS
GFP encoding lentivirus (LentiArray™ CRISPR negative control Lentivirus, human, non-targeting with GFP) was introduced at the MOI of 10 with polybrene
MOS and virus were spun in the centrifuge at 200×g. 37 C for 2 hours
cells were transferred to a plate and incubated overnight
the plate was washed to remove any extracellular virus
After two days, the MOSs were imaged. Afterwards, single cells were acquired via MOS dissociation and fixed with 4% paraformaldehyde solution, followed by flow cytometry to confirm successful transduction.

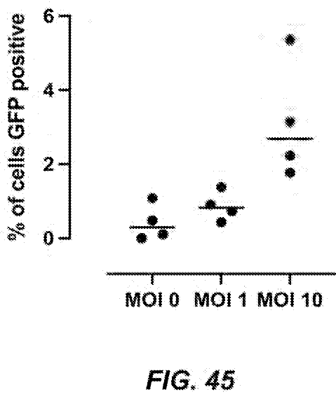

FIG. 45 shows the results of lentiviral vector-based delivery of components into a MOS. Cells are editable using viruses as a delivery mechanism.

### Example 12: Electroporation Methods to Deliver Components into a MOS

A GFP plasmid (Lonza pMaxGFP) was used as a read out to test the electroporation methods used to deliver components into a MOS.

The DNA plasmid containing GFP is large, several times larger than the CRISPR/Cas9 complex, and so it was expected to be harder to deliver into a MOS, but had the advantage of providing a visual readout that could be quantitated by simple methods.

The GFP plasmid was delivered to MOSs according to the following protocol:
One day prior to nucleofection MOSs were generated with CRC patient derived organoids cells in 70% MATRIGEL. 10-15 cells per MOS
80 reactions were conducted in parallel during the initial optimization experiment, with five different Primary Cell 4D-Nucleofector Solutions P1-P5 in combination with 15 different Nucleofector Programs (i.e., CA-137, CM-138, CM-137, CM-150, DN-100, DS-138, DS-137, DS-130, DS-150, DS-120, EH-100, EO-100, EN-138, EN150, EW-113) plus one control.
Each reaction consisted of 20 uL P1/P2/P3/P4/P5 4D-Nucleofector X Solution, 0.4 ug pmaxGFP Vector and 75-100 MOSs. The reactions were transferred to corresponding wells in the 16-well Nucleocuvette Strips followed by nucleofection on the 4D-Nucleofector X Unit with pre-set Nucleofector Programs for each well.
After run completion, the Nucleocuvette Strips were incubated for 10 min at room temperature.
The MOSs were resuspended with 180 uL pre-warmed medium by gently pipetting up and down two to three times, and replated in a 96-well plate.
After 4 days, the MOSs were imaged, followed by MOS dissociation and flow cytometry to confirm the efficiency of the nucleofection.

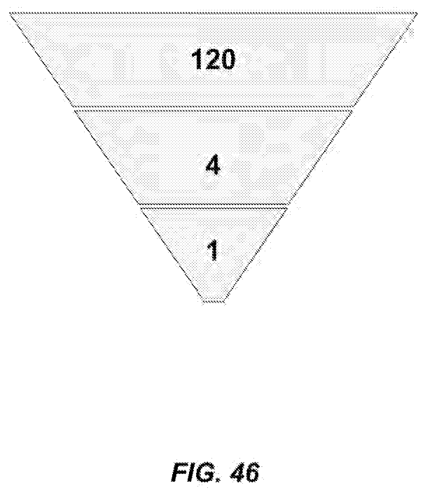

As shown in FIG. 46, 120 different electroporation conditions were tested. The conditions were based on eight different buffers and 15 different pulse conditions.

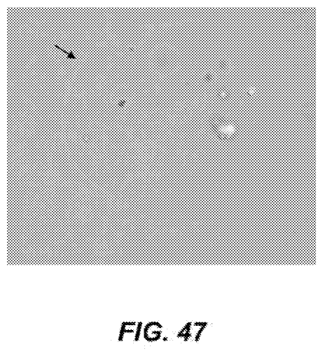

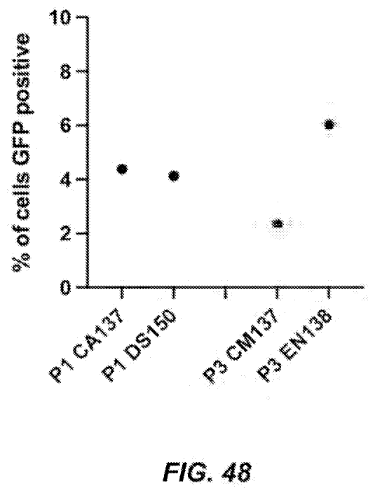

Four combinations of buffer and pulse conditions were effective and individual MOSs expressed GFP as shown in FIG. 47. FIG. 48 shows the percentage of cells that took up the GFP plasmid and expressed GFP. The pulse conditions and buffers are shown on the X axis.

### Example 13: Gene Editing in MOS Using CRISPR/Cas9 Complex Components

Gene editing in MOSs was assessed by delivering CRISPR/Cas9 ribonucleoprotein by electroporation.

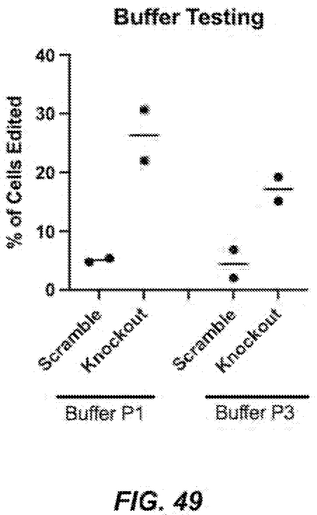

FIG. 49 shows the results of testing different buffers. The P1 buffer showed a greater % of edited cells compared to the P3 buffer.

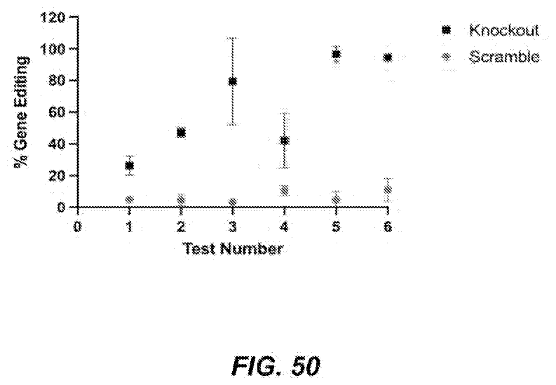

FIG. 50 illustrates the effects of different parameters on gene editing and MOS stability. Test number 6 is a repeat of test number 5.

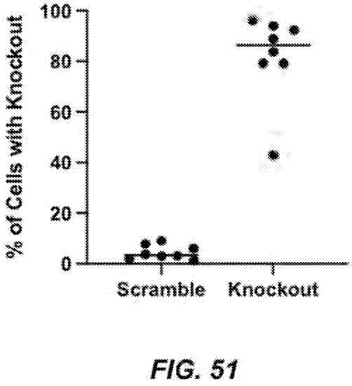

FIG. 51 illustrates the results of gene editing in MOSs using the following protocol:

PDOs were split into new domes 3 days prior to use

MOSs were generated with CRC patient derived organoids cells in 60% MATRIGEL, 10 cells per MOS

MOSs were plated overnight prior to electroporation

### Next Day

CRISPR/Cas9 ribonucleoprotein complexes (RNPs) were generated by combining the following, in order:

16.4 ul P1 buffer/sample

3.6 ul supplement/sample

2.2 ul of sgRNA at 30 pmol/ul (B2M-CGGAGCGAGAGAGCACAGCG, GGCCGAGAUGUCUCGCUCCG, ACUCACGCUGGAUAGCCUCC)

1.6 ul of Cas 9 (Synthego, 20 pmol/ul)

20 minute pre-incubation to form RNPs before use→Warmed to 37 C before adding to MOSs

If doing a longitudinal study, enough RNPs were made for all time points.

RNPs were saved at 4 C for use each day in the time course, and warmed to RT before electroporation

MOSs were Harvested

Harvested MOSs were centrifuged at 200×g for 3 mins at 25 C. The centrifuge should not be used at 4 C.

As much supernatant as possible was removed→if samples were to be used with different RNPs then they were resuspended

MOSs were counted on the EVOS cell imaging system and 100 MOS added per well

MOSs were resuspended with 20 ul of RNP solution (for 16 well strip tubes)

MOSs were incubated for 20 mins at 37 C

MOSs were electroporated with setting CA-137

6 days after MOS generation

The samples were harvested, digested with tryple and stained with relevant antibodies-live/dead V450 and APC B2M (1:200 dilution) for 30 mins on ice.
Flow cytometry was run to analyze results. A standard protocol was used.

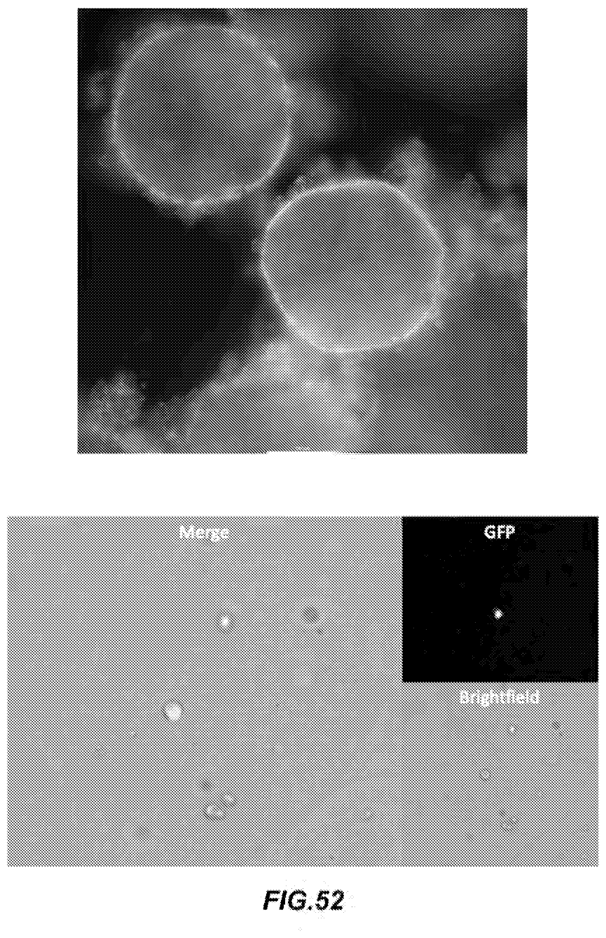

FIG. 52 shows Cas9-GFP on the edges of MOS.

The green CRISPR/Cas9 signal throughout the MOS indicates that CRISPR/Cas9 penetrates throughout the MOS within 20 minutes of adding the protein. Except for the high concentration at the borders of the MOS, there are no clear rings of green that indicate a large concentration gradient of CRISPR/Cas9 within the MOS. This data means that gene editing can occur in any cells within the MOS regardless of location.

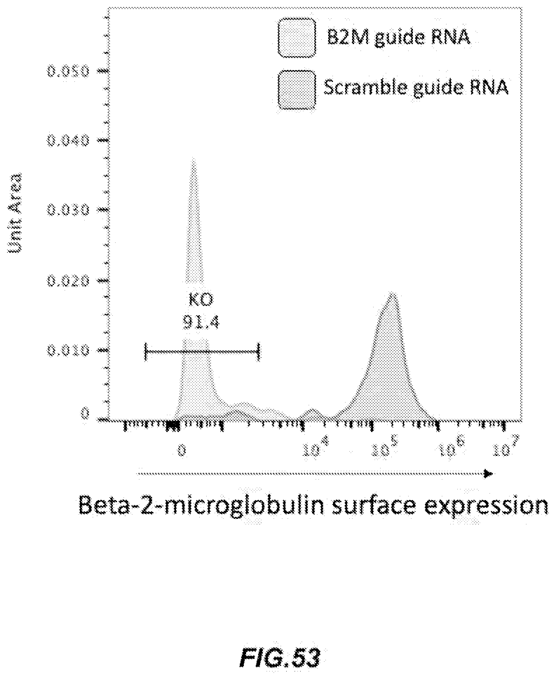

FIG. 53 shows analysis of RNP MOS delivery.

As described above, cells in MOS were transformed using a CRISPR/Cas9 complex with a guide RNA targeting Beta-2-microglobulin. The purpose of this experiment was to knockout the gene in the cells for downstream evaluation. The cells were assessed for the surface expression of Beta-2-microglobulin by flow cytometry after transformation to assess how many cells lost the B2M protein. In this representative example, 91.4% of the cells had no B2M protein expression. The loss of B2M indicates both copies of the gene were successfully edited. The Scramble guide RNA sample used a guide RNA that did not target Beta-2-microglobulin in the CRISPR/Cas9 complex and shows specificity of the knockout to the specific guide RNAs used.

### CONCLUSION

The methods allow delivery of CRISPR-CAS gene editing components to one or more cells within MOS, and that editing efficiencies of about 80%, greater than 80%, or greater than 90% are obtainable.

Although certain conditions for electroporating MOS are described, electroporation could be accomplished in any traditionally suitable setting for electroporating cells. For example, any suitable cuvette or similar container that can hold MOS and is configured to receive electricity for electroporation could be used.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control/perform any of the steps, including but not limited to: displaying, communicating with the user, analyzing, modifying parameters (including timing, frequency, intensity, etc.), determining, alerting, or the like.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting. For example, as used herein, the singular forms “a”, “an” and “the” are intended to include the plural forms as well, unless the context clearly indicates otherwise. It will be further understood that the terms “comprises” and/or “comprising.” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”,

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed herein could be termed a second feature/element, and similarly, a second feature/element discussed herein could be termed a first feature/element without departing from the teachings of the present invention.

Throughout this specification, unless the context requires otherwise, the word “comprise”, and embodiments such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any embodiment calculated to achieve the same purpose may be substituted for the specific embodiments shown. This disclosure is intended to cover any and all adaptations or embodiments of various embodiments. Combinations of the above embodiments, and other embodiments not specifically described herein, will be apparent to those of skill in the art upon reviewing the above description.

1-60. (canceled)

61. A method of editing DNA or RNA in one or more cells within a droplet, the method comprising:
a) delivering one or more CRISPR/Cas complex components into the droplet under conditions that maintain cell viability; and
b) incubating the droplet under conditions suitable for CRISPR/Cas mediated DNA or RNA editing.

62. The method according to claim 61 wherein the one or more CRISPR/Cas complex components is a ribonucleoprotein.

63. The method according to claim 61, wherein the one or more components are one or more CRISPR/Cas9 complex components.

64. The method according to claim 61, wherein the one or more cells are diseased cells.

65. The method according to claim 61, wherein the one or more cells are non-diseased cells.

66. The method according to claim 61, wherein the droplet has a diameter from 100 uM to 500 uM.

67. The method according to claim 61, wherein the droplet has a diameter of about 260 uM.

68. The method according to claim 61, wherein the one or more CRISPR/Cas complex components are delivered into the droplet by viral vector-based delivery.

69. The method according to claim 68, wherein the viral vector is a lentiviral vector.

70. The method according to claim 61, wherein the one or more CRISPR/Cas complex components are delivered into the droplet by lipid-based delivery.

71. The method according to claim 61, wherein the one or more CRISPR/Cas complex components are delivered into the droplet by electroporation.

72. The method according to claim 71, wherein the one or more CRISPR/Cas complex components are incubated with the droplet for about 1 minute to 60 minutes prior to electroporation.

73. The method according to claim 72, wherein the one or more CRISPR/Cas complex components are incubated with the droplet for about 20 minutes prior to electroporation.

74. The method according to claim 71, wherein the one or more CRISPR/Cas complex components are incubated with the droplet at a temperature of 20 to 40 degrees centigrade prior to electroporation.

75. The method according to claim 74, wherein the one or more CRISPR/Cas complex components are incubated with the droplet at a temperature of about 37 degrees centigrade prior to electroporation.

76. The method according to claim 61, wherein the method comprises dispersing the droplet into a multiwell plate prior to delivering the one or more CRISPR/Cas complex components into the droplet.

77. The method according to claim 61, wherein a 3D microenvironment develops within the droplet.

78. The method according to claim 61, wherein an organoid or a tissue model develops within the droplet.

79. The method according to claim 61, wherein the DNA or RNA editing efficiency is from 50% to 99%.

80. The method according to claim 79, wherein the DNA or RNA editing efficiency is about 80%.

81. The method according to claim 61, wherein the DNA or RNA editing efficiency is greater than 80%.

82. The method according to claim 81, wherein the DNA or RNA editing efficiency is greater than 90%.

83. A droplet comprising: (i) one or more cells, and (ii) one or more CRISPR/Cas complex components.

84. The droplet according to claim 83, wherein one or more of the CRISPR/Cas complex components is a ribonucleoprotein.

85. The droplet according to claim 83, wherein the one or more cells are diseased cells.

86. The droplet according to claim 83, wherein the one or more cells are non-diseased cells

87. The droplet according to claim 83, wherein the droplet has a diameter from 100 uM to 500 uM.

88. The droplet according to claim 87, wherein the droplet has a diameter of about 260 uM.
